(19) United States
(12) Patent Application Publication
(10) Pub. No.: US 2025/0099443 A1
(43) Pub. Date: Mar. 27, 2025

(54) METHODS FOR SYNTHESIZING ALEUTIANAMINE AND ANALOGS THEREOF

(71) Applicant: California Institute of Technology, Pasadena, CA (US)

(72) Inventors: Hao Yu, Pasadena, CA (US); Zachary P. Sercel, Haifa (IL); Samir P. Rezgui, Pasadena, CA (US); Jonathan Farhi, Pasadena, CA (US); Brian M. Stoltz, San Marino, CA (US); Scott C. Virgil, Pasadena, CA (US)

(21) Appl. No.: 18/884,373

(22) Filed: Sep. 13, 2024

Related U.S. Application Data
(60) Provisional application No. 63/538,754, filed on Sep. 15, 2023.

(51) Int. Cl. A61K 31/4375; A61K 31/437; A61P 35/00; C07D 471/06; C07D 519/00

(52) U.S. Cl. CPC A61K 31/4375; A61K 31/437; A61P 35/00; C07D 471/06; C07D 519/00

(57) ABSTRACT

Disclosed herein are analogs and derivatives of aleutianamine and methods of making the same. Also disclosed herein are methods of treating pancreatic cancer comprising administration of aleutianamine or its analogs.

### RELATED APPLICATIONS

This application claims the benefit of priority to U.S. Provisional Application No. 63/538,754, filed Sep. 15, 2023; the entire contents of which are incorporated herein by reference.

### STATEMENT OF GOVERNMENT SUPPORT

This invention was made with government support under Grant No. CHE2247315 awarded by the National Science Foundation. The government has certain rights in the invention.

### BACKGROUND

Cancer-related illnesses are the second leading cause of death in the United States behind only heart disease. Pancreatic cancer is the third leading cause of cancer death and is projected to be the second deadliest cancer by 2040, exemplified by a dismal 12% five-year survival rate for patients with the disease. These alarming statistics can be attributed to difficulties in early disease detection, the lack of common genetic mutations associated with the disease, and overall ineffective treatment options. Despite advances in new therapeutics for pancreatic cancer, patient survival has only marginally increased in the last several decades.

Historically, natural products have contributed significantly toward drug discovery and novel therapeutics, particularly in the areas of cancer and infectious disease. Indeed, several of the state-of-the-art therapies for pancreatic cancer are natural products or natural product derivatives. Aleutianamine (1), isolated in 2019 by Hamann and coworkers, is a marine-derived alkaloid that possesses potent and selective cytotoxicity toward solid tumor cell lines. Most notably, it displays a 25 nM IC₅₀ against the human pancreatic adenocarcinoma cell line PANC-1. This potency is over 160 times greater than that of the FDA-approved chemotherapeutic agent gemcitabine, demonstrating the therapeutic potential of the natural product. However, access to aleutianamine analogs would enable study of its structure-activity relationship, and potentially lead to a clinically viable derivative. In view of the foregoing, there is an unmet need to develop analogs of aleutianamine.

### Summary of the Invention

### BRIEF DESCRIPTION OF THE DRAWINGS

FIG. 1 depicts overlayed spectra of synthetic and reported ¹H NMR spectra of aleutianamine (400 MHz).

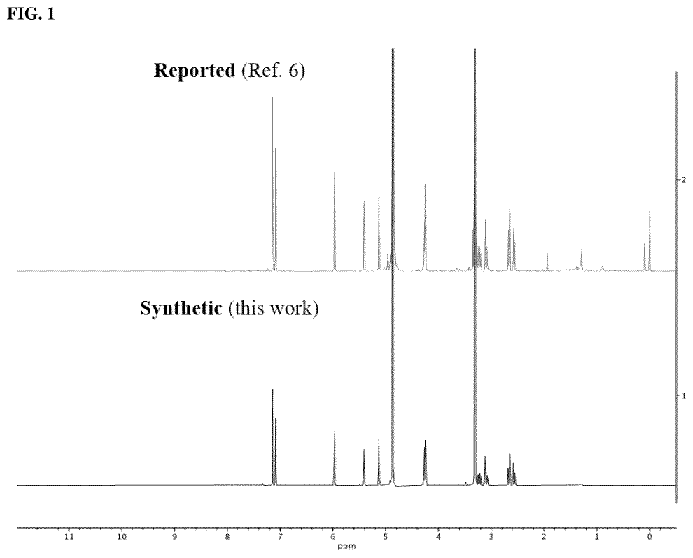

FIG. 2 depicts overlayed spectra of synthetic and reported ¹³C NMR spectra of aleutianamine (100 MHz).

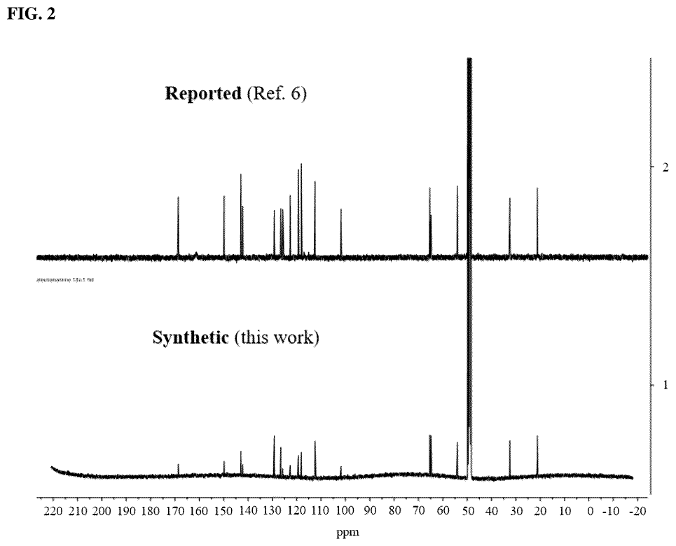

### DETAILED DESCRIPTION OF THE INVENTION

Aleutianamine is a recently isolated pyrroloiminoquinone natural product that displays potent and selective biological activity toward human pancreatic cancer cells with an IC₅₀=25 nM against PANC-1, making it a potential candidate for therapeutic development. The present disclosure relates to a synthetic approach to aleutianamine wherein the unique [3.3.1] ring system and tertiary sulfide of this alkaloid were constructed via a novel palladium-catalyzed dearomative thiophene functionalization. Other highlights of the synthesis include a palladium-catalyzed decarboxylative pinacol-type rearrangement of an allylic carbonate to install a ketone and a late-stage oxidative amination. This concise and convergent strategy will enable access to analogues of aleutianamine and further investigation of the biological activity of this unique natural product.

Aleutianamine (1) belongs to the pyrroloiminoquinone alkaloid family of natural products, defined by their conserved central planar, tricyclic ring system. These natural products have received significant attention from the synthetic community due to their complex molecular frameworks and broad biological activities. Structurally, aleutianamine (1) possesses a unique heptacyclic ring system which consists of a pyrroloiminoquinone unit, a bridged azabicyclo[3.3.1]nonane ring system substituted with a congested tertiary alkyl sulfide and an alkenyl bromide, and another bridging thioaminal linkage (Scheme 1A). The multi-bridged ring system of the natural product bears three stereocenters and is highly strained due to extensive unsaturation. The congested sulfide, the potentially labile thioaminalmoiety, and the remote alkenyl bromide represent considerable synthetic challenges.

Aleutianamine (1) is proposed to arise biosynthetically from either makaluvamine F (2) or discorhabdin B (3) (Scheme 1A). Recently, Tokuyama accomplished the first total synthesis of aleutianamine (1) by a biomimetic approach wherein a discorhabdin B analog underwent a cationic rearrangement to produce the ring system of aleutianamine (1), supporting the proposed biosynthetic pathway.

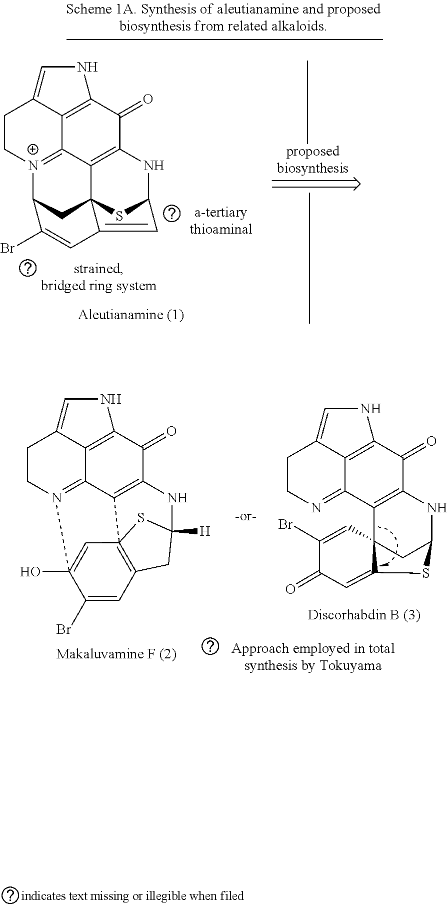

The present disclosure relates to an alternative non-biomimetic approach centered around formation of the bridging [3.3.1] ring system followed by late-stage arene oxidation, a strategy that is unique in comparison to previous pyrroloiminoquinone syntheses (Scheme 1B).

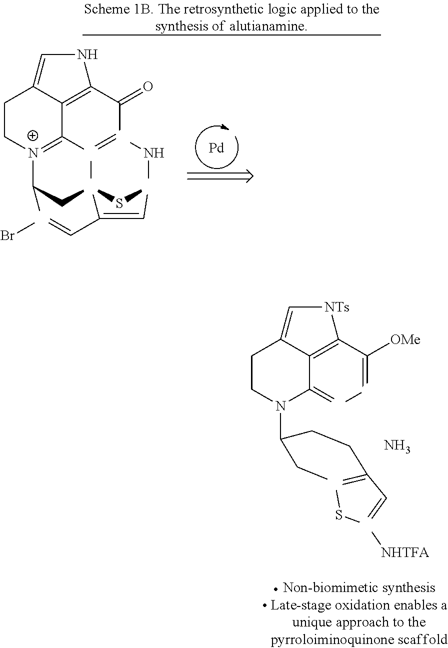

The dearomative arylation of a thiophene was envisioned as a key transformation to enable this strategy, as this reaction would construct the bridging [3.3.1] ring system and congested tertiary bridgehead sulfide in a single synthetic step (Scheme 1C).

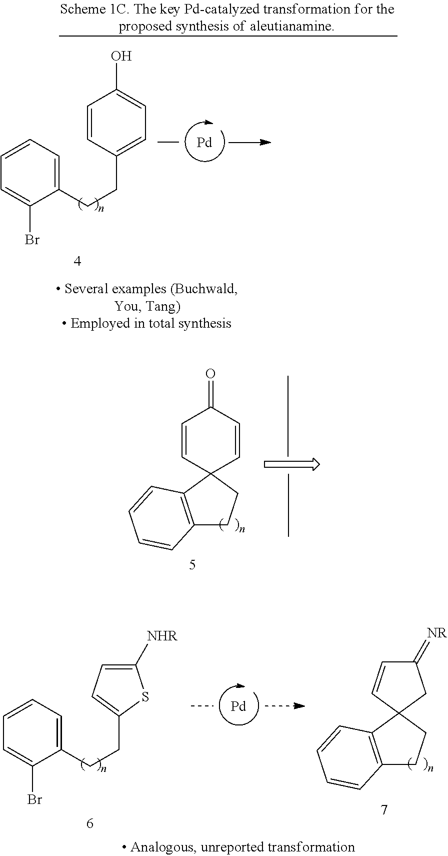

The transformation was inspired by previous reports of dearomative phenol cross-couplings, but the analogous transformation of thiophenes has yet to be reported. Retrosynthetically, oxidation state adjustment and cleavage of the aryl C—N bond of aleutianamine (1) would lead back to thiolactone 8 (Scheme 2).

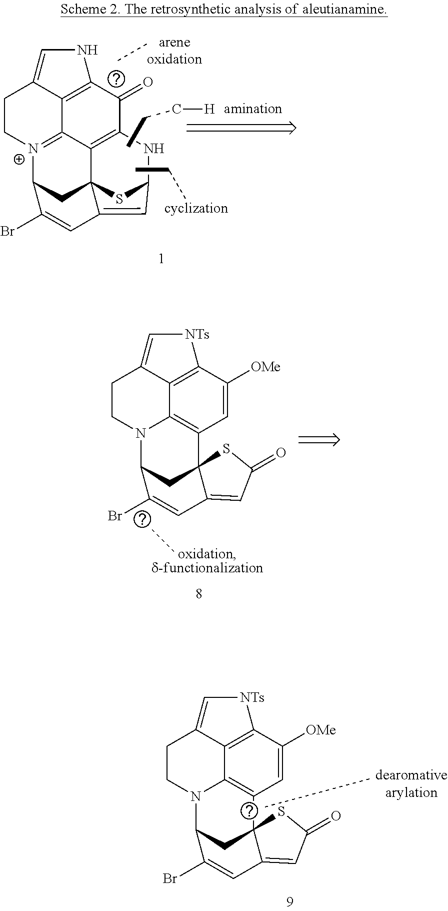

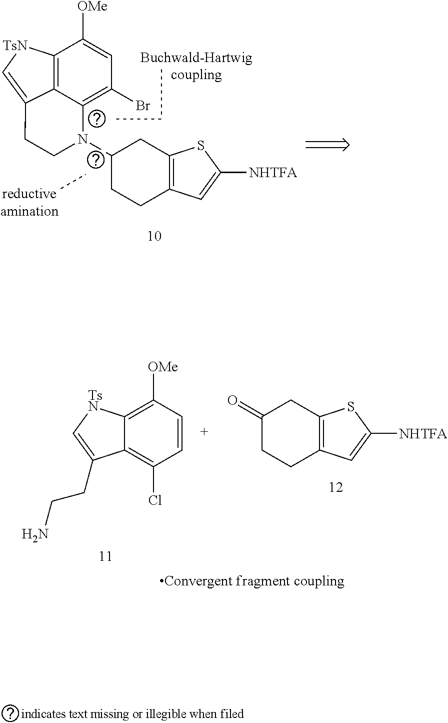

This intermediate would arise from partially saturated thiolactone 9 by installation of the vinyl bromide. Retrosynthetic cleavage of the Csp²-Csp³ bond of the [3.3.1] ring system by the proposed dearomative arylation simplifies the target to aryl bromide 10, which could be rapidly prepared in a convergent fashion from tryptamine 11 and aminothiophene 12.

These studies commenced with the Fischer Indole Synthesis of tryptophol 14 from known arylhydrazine 13 and dihydrofuran (Scheme 3A). A three-step sequence involving a Mitsunobu reaction with DPPA, subsequent N-tosylation, and Staudinger reduction yielded protected tryptamine 11.

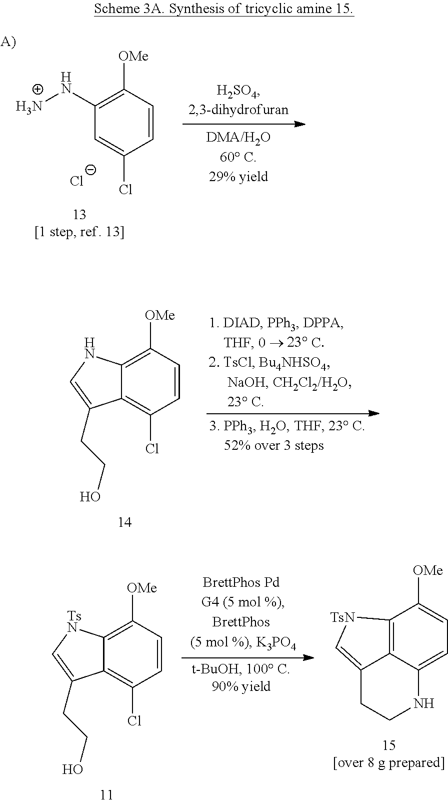 [SMILES: COc1ccc(Cl)cc1N[NH3+] ; [Cl-] ; COc1ccc(Cl)c2c(CCO)c[nH]c12 ; COc1ccc(Cl)c2c(CCO)cn(S(=O)(=O)c3ccc(C)cc3)c12 ; COc1ccc2c3c(cn(S(=O)(=O)c4ccc(C)cc4)c13)CCN2]

Finally, intramolecular Buchwald-Hartwig amination provided tricyclic aniline 15. The synthesis of thiophene coupling fragment 12 (Scheme 3B) began with aminothiophene 16, which was prepared in one step from 1,4-cyclohexanedione monoethylene ketal via Gewald aminothiophene synthesis. Saponification and decarboxylation served to remove the ester group, and the resulting amine was readily protected as the trifluoroacetamide.

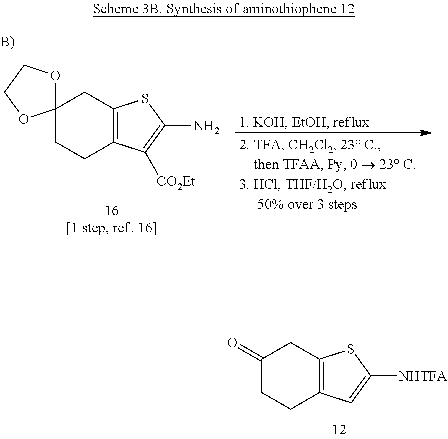

Subsequent ketal cleavage afforded ketothiophene coupling partner 12. Coupling of tricyclic aniline 15 with ketothiophene 12 was achieved by employing indium hydride-mediated reductive amination conditions developed by Yang (Scheme 4). Subsequent bromination yielded cyclization precursor 10. Treatment of bromoaniline 10 with Pd(dba)₂ and XPhos in the presence of base led to the desired dearomative cyclization with concomitant cleavage of the trifluoroacetamide to yield free thioimidate 17.

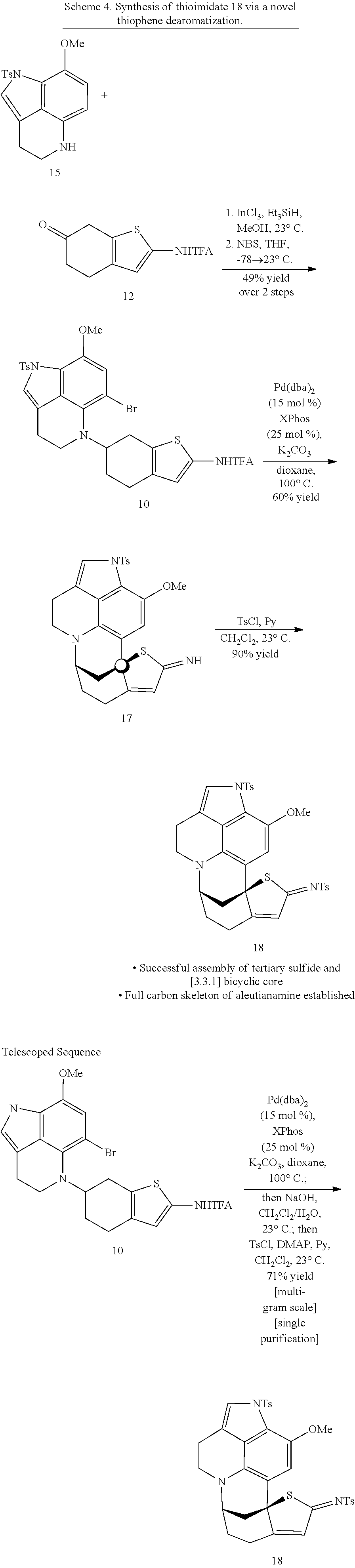

In addition to completing the carbon skeleton of aleutianamine (1) and assembling the tertiary alkyl sulfide, this reaction constitutes the first dearomative arylation of a thiophene derivative to date. The product of this coupling (17) was then N-tosylated to provide thioimidate 18—the N-tosyl group proved critical for further functionalization, as free thioimidate 17 was recalcitrant to hydrolysis and other electron-withdrawing groups were excessively labile. To circumvent the challenging purification of free thioimidate 17, the dearomative arylation and tosylation steps were telescoped to provide an improved yield of tosyl thioimidate 18 on multi-gram scale. Subjection of intermediate 18 to aqueous alkaline conditions led to hydrolysis of the tosyl thioimidate, yielding thiobutenolide 9 (Scheme 5).

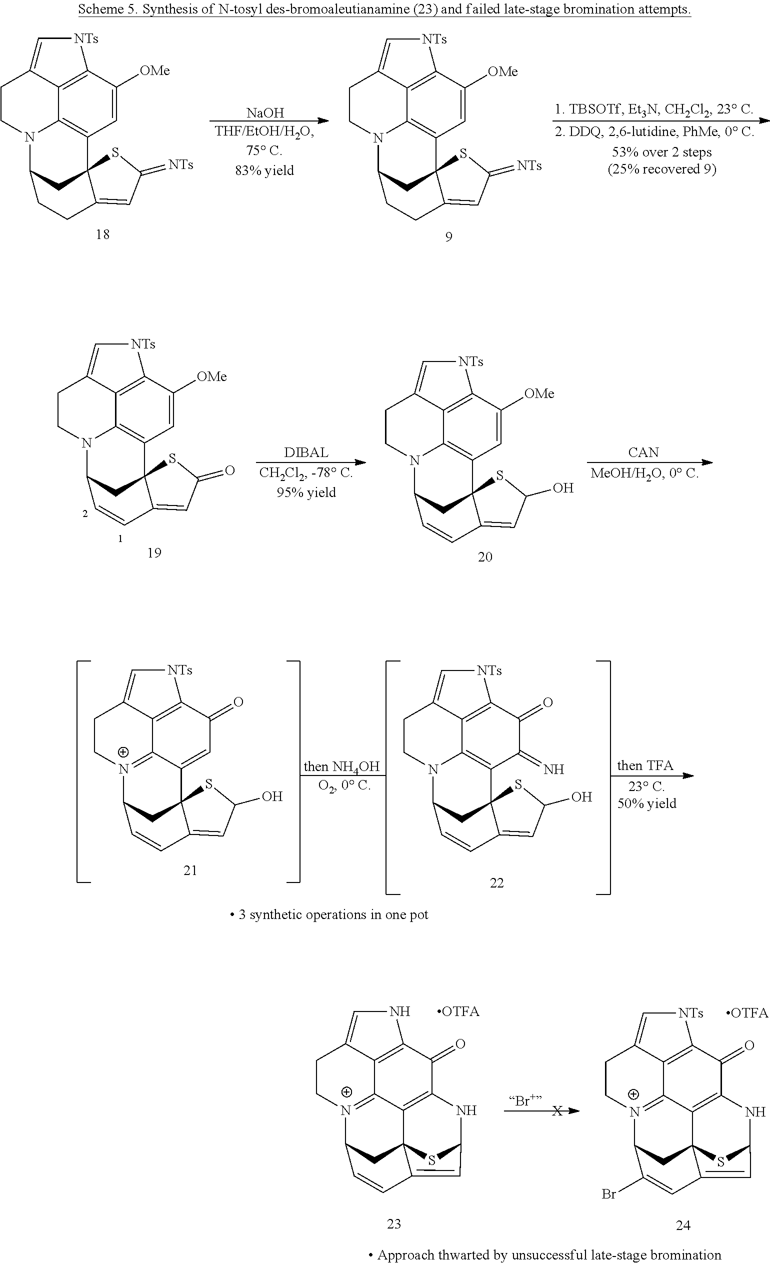

While a variety of standard conditions failed to promote vinylogous desaturation of this intermediate (9), soft enolization with TBSOTf afforded an intermediate silyl ketene thioacetal that was treated with DDQ to provide diene 19, the structure of which was confirmed by X-ray crystallography. Reasoning that the electronics of diene 19 would promote bromination at the undesired C1 position, installation of the alkenyl bromide was delayed until the final stage of the synthesis. Thus, DIBAL reduction of the thiolactone smoothly provided thiolactol 20. Treatment with CAN oxidized the arene to the desired pyrroloiminoquinone 21, and addition of aqueous ammonia effected amination and aerobic oxidation to deliver pyrroloiminoquinone 22. Finally, addition of TFA led to dehydrative cyclization to yield thioaminal 23, which bears the full ring system of aleutianamine (1). Unfortunately, direct bromination of N-tosyl desbromoaleutianamine (23) was unsuccessful; all surveyed bromination conditions led to decomposition or undesired regioselectivity, although the surveyed bromination conditions (four in total) were not exhaustive.

To circumvent the unsuccessful late-stage bromination, an attempt was made to increase the oxidation state at C2 on diene 19, which would provide a functional handle for bromination. However, incorporation of a suitable functional handle proved to be a significant challenge. Installation of a ketone or an equivalent thereof at the C2 position was attempted by allylic oxidation, thia-Michael addition and Pummerer rearrangement, and organosilane or organoborane 1,6-addition. Additionally, incorporation of an oxidation handle into aminothiophene coupling fragment 12 proved unsuccessful. Ultimately, ketone installation was achieved via an unconventional sequence. Beginning with diene 19, γ,δdihydroxylation with OsO₄ provided diol 25, which was readily advanced to carbonate 26 (Scheme 6).

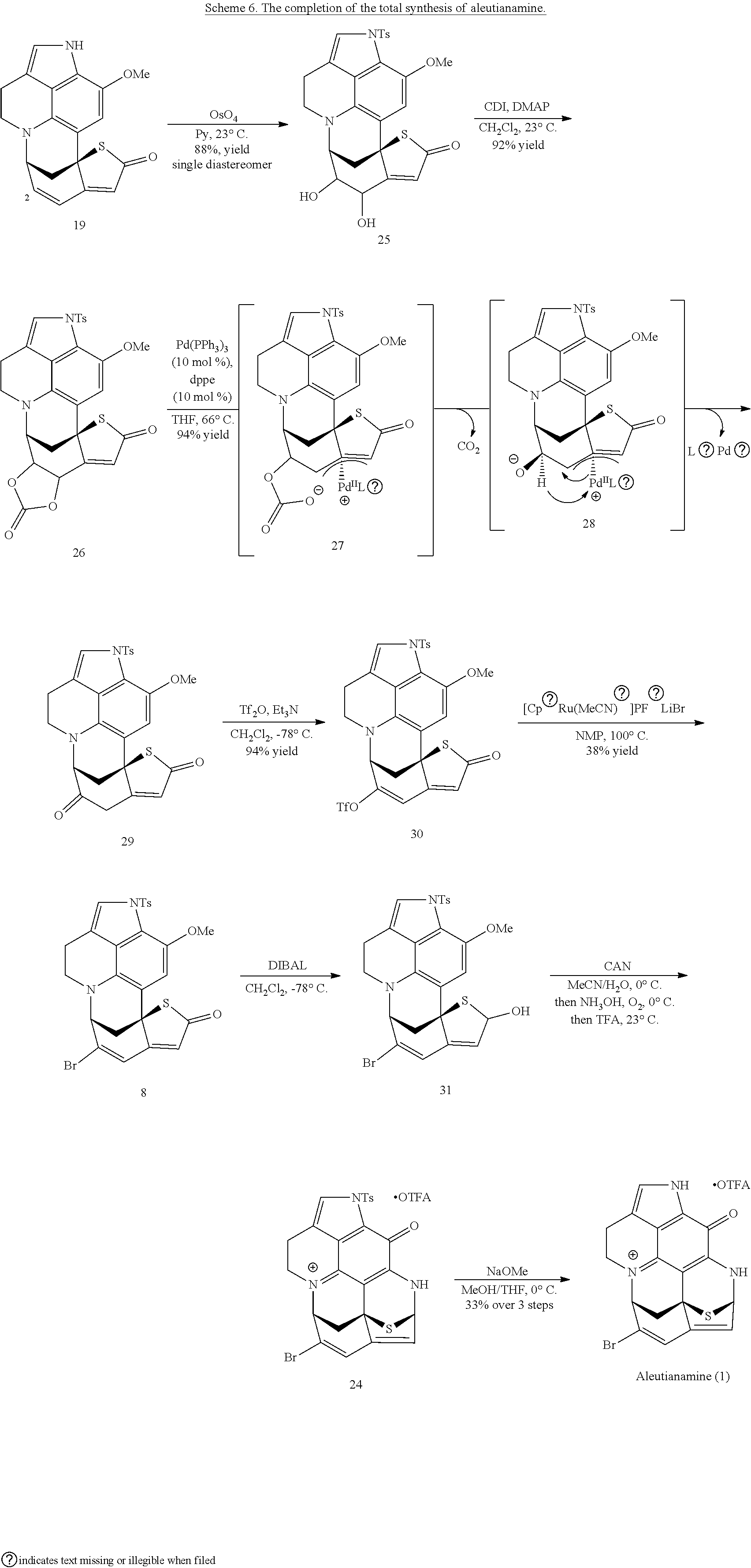

Treatment with Pd(0) and dppe led to a decarboxylative pinacol-type rearrangement, presumably via putative π-allyl Pd(II) intermediate 27, which could undergo decarboxylation to afford 28, and subsequent β-hydride elimination and tautomerization to afford desired ketone 29. This represents the first palladium-catalyzed decarboxylative pinacol-type rearrangement of allylic carbonates. Conversion to enol triflate 30 was followed by Shirakawa and Hayashi's Ru-catalyzed triflate-halogen exchange to provide desired alkenyl bromide 8. Finally, in an analogous fashion to the synthesis of des-bromo compound 23, 1,2-reduction with DIBAL followed by oxidative amination and cyclization yielded penultimate intermediate 24, and tosyl group cleavage with NaOMe afforded aleutianamine (1) in a longest linear sequence of 20 steps.

This total synthesis represents a non-biomimetic synthetic approach to aleutianamine (1). Key to the synthetic approach was the Pd-catalyzed intramolecular dearomative arylation of an aminothiophene, ketone installation by the Pd-catalyzed pinacol-type rearrangement of a cyclic carbonate, and late-stage arene oxidative amination. Efforts to prepare analogues of aleutianamine by related sequences and to establish a structure-activity relationship against biologically relevant cancer cell lines are ongoing.

### Pharmaceutical Compositions

The compositions and methods of the present invention may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, oils such as olive oil, or injectable organic esters. In preferred embodiments, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection or the like. The composition can also be present in a transdermal delivery system, e.g., a skin patch. The composition can also be present in a solution suitable for topical administration, such as a lotion, cream, or ointment.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation or pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); subcutaneously; transdermally (for example as a patch applied to the skin); and topically (for example, as a cream, ointment or spray applied to the skin). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans; and other mammals such as equines, cattle, swine, sheep, cats, and dogs; poultry; and pets in general.

In certain embodiments, compounds of the invention may be used alone or conjointly administered with another type of therapeutic agent.

The present disclosure includes the use of pharmaceutically acceptable salts of compounds of the invention in the compositions and methods of the present invention. In certain embodiments, contemplated salts of the invention include, but are not limited to, alkyl, dialkyl, trialkyl or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn or other metal salts. In certain embodiments, contemplated salts of the invention include, but are not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, 1-ascorbic acid, 1-aspartic acid, benzenesulfonic acid, benzoic acid, (+)-camphoric acid, (+)-camphor-10-sulfonic acid, capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, d-glucoheptonic acid, d-gluconic acid, d-glucuronic acid, glutamic acid, glutaric acid, glycerophosphoric acid, glycolic acid, hippuric acid, hydrobromic acid, hydrochloric acid, isobutyric acid, lactic acid, lactobionic acid, lauric acid, maleic acid, 1-malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, 1-pyroglutamic acid, salicylic acid, sebacic acid, stearic acid, succinic acid, sulfuric acid, 1-tartaric acid, thiocyanic acid, p-toluenesulfonic acid, trifluoroacetic acid, and undecylenic acid acid salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

### Definitions

Unless otherwise defined herein, scientific and technical terms used in this application shall have the meanings that are commonly understood by those of ordinary skill in the art. Generally, nomenclature used in connection with, and techniques of, chemistry, cell and tissue culture, molecular biology, cell and cancer biology, neurobiology, neurochemistry, virology, immunology, microbiology, pharmacology, genetics and protein and nucleic acid chemistry, described herein, are those well known and commonly used in the art.

The methods and techniques of the present disclosure are generally performed, unless otherwise indicated, according to conventional methods well known in the art and as described in various general and more specific references that are cited and discussed throughout this specification. See, e.g. “Principles of Neural Science”, McGraw-Hill Medical, New York, N.Y. (2000); Motulsky, “Intuitive Biostatistics”, Oxford University Press, Inc. (1995); Lodish et al., “Molecular Cell Biology, 4th ed.”, W. H. Freeman & Co., New York (2000); Griffiths et al., “Introduction to Genetic Analysis, 7th ed.”, W. H. Freeman & Co., N.Y. (1999); and Gilbert et al., “Developmental Biology, 6th ed.”, Sinauer Associates, Inc., Sunderland, MA (2000).

Chemistry terms used herein, unless otherwise defined herein, are used according to conventional usage in the art, as exemplified by “The McGraw-Hill Dictionary of Chemical Terms”, Parker S., Ed., McGraw-Hill, San Francisco, C.A. (1985).

All of the above, and any other publications, patents and published patent applications referred to in this application are specifically incorporated by reference herein. In case of conflict, the present specification, including its specific definitions, will control.

The term “agent” is used herein to denote a chemical compound (such as an organic or inorganic compound, a mixture of chemical compounds), a biological macromolecule (such as a nucleic acid, an antibody, including parts thereof as well as humanized, chimeric and human antibodies and monoclonal antibodies, a protein or portion thereof, e.g., a peptide, a lipid, a carbohydrate), or an extract made from biological materials such as bacteria, plants, fungi, or animal (particularly mammalian) cells or tissues. Agents include, for example, agents whose structure is known, and those whose structure is not known.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats).

“Treating” a condition or patient refers to taking steps to obtain beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treatment” can also mean prolonging survival as compared to expected survival if not receiving treatment.

The term “preventing” is art-recognized, and when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount.

“Administering” or “administration of” a substance, a compound or an agent to a subject can be carried out using one of a variety of methods known to those skilled in the art. For example, a compound or an agent can be administered, intravenously, arterially, intradermally, intramuscularly, intraperitoneally, subcutaneously, ocularly, sublingually, orally (by ingestion), intranasally (by inhalation), intraspinally, intracerebrally, and transdermally (by absorption, e.g., through a skin duct). A compound or agent can also appropriately be introduced by rechargeable or biodegradable polymeric devices or other devices, e.g., patches and pumps, or formulations, which provide for the extended, slow or controlled release of the compound or agent. Administering can also be performed, for example, once, a plurality of times, and/or over one or more extended periods.

Appropriate methods of administering a substance, a compound or an agent to a subject will also depend, for example, on the age and/or the physical condition of the subject and the chemical and biological properties of the compound or agent (e.g., solubility, digestibility, bioavailability, stability and toxicity). In some embodiments, a compound or an agent is administered orally, e.g., to a subject by ingestion. In some embodiments, the orally administered compound or agent is in an extended release or slow release formulation, or administered using a device for such slow or extended release.

As used herein, the phrase “conjoint administration” refers to any form of administration of two or more different therapeutic agents such that the second agent is administered while the previously administered therapeutic agent is still effective in the body (e.g., the two agents are simultaneously effective in the patient, which may include synergistic effects of the two agents). For example, the different therapeutic compounds can be administered either in the same formulation or in separate formulations, either concomitantly or sequentially. Thus, an individual who receives such treatment can benefit from a combined effect of different therapeutic agents.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated, such as cancer or MDS. The skilled worker can readily determine the effective amount for a given situation by routine experimentation.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as where the alkyl is not substituted.

It is understood that substituents and substitution patterns on the compounds of the present invention can be selected by one of ordinary skilled person in the art to result chemically stable compounds which can be readily synthesized by techniques known in the art, as well as those methods set forth below, from readily available starting materials. If a substituent is itself substituted with more than one group, it is understood that these multiple groups may be on the same carbon or on different carbons, so long as a stable structure results.

As used herein, the term “optionally substituted” refers to the replacement of one to six hydrogen radicals in a given structure with the radical of a specified substituent including, but not limited to: hydroxyl, hydroxyalkyl, alkoxy, halogen, alkyl, nitro, silyl, acyl, acyloxy, aryl, cycloalkyl, heterocyclyl, amino, aminoalkyl, cyano, haloalkyl, haloalkoxy, —OCO—CH₂—O-alkyl, —OP(O)(O-alkyl)₂ or —CH₂—OP(O)(O-alkyl)₂. Preferably, “optionally substituted” refers to the replacement of one to four hydrogen radicals in a given structure with the substituents mentioned above. More preferably, one to three hydrogen radicals are replaced by the substituents as mentioned above. It is understood that the substituent can be further substituted.

As used herein, the term “alkyl” refers to saturated aliphatic groups, including but not limited to C₁-C₁₀ straight-chain alkyl groups or C₁-C₁₀ branched-chain alkyl groups. Preferably, the “alkyl” group refers to C₁-C₆ straight-chain alkyl groups or C₁-C₆ branched-chain alkyl groups. Most preferably, the “alkyl” group refers to C₁-C₄ straight-chain alkyl groups or C₁-C₄ branched-chain alkyl groups. Examples of “alkyl” include, but are not limited to, methyl, ethyl, 1-propyl, 2-propyl, n-butyl, sec-butyl, tert-butyl, 1-pentyl, 2-pentyl, 3-pentyl, neo-pentyl, 1-hexyl, 2-hexyl, 3-hexyl, 1-heptyl, 2-heptyl, 3-heptyl, 4-heptyl, 1-octyl, 2-octyl, 3-octyl or 4-octyl and the like. The “alkyl” group may be optionally substituted.

The term “acyl” is art-recognized and refers to a group represented by the general formula hydrocarbylC(O)—, preferably alkylC(O)—.

The term “acylamino” is art-recognized and refers to an amino group substituted with an acyl group and may be represented, for example, by the formula hydrocarbylC(O)NH—.

The term “acyloxy” is art-recognized and refers to a group represented by the general formula hydrocarbylC(O)O—, preferably alkylC(O)O—.

The term “alkoxy” refers to an alkyl group having an oxygen attached thereto. Representative alkoxy groups include methoxy, ethoxy, propoxy, tert-butoxy and the like.

The term “alkoxyalkyl” refers to an alkyl group substituted with an alkoxy group and may be represented by the general formula alkyl-O-alkyl.

The term “alkyl” refers to saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In preferred embodiments, a straight chain or branched chain alkyl has 30 or fewer carbon atoms in its backbone (e.g., C₁₋₃₀ for straight chains, C₃₋₃₀ for branched chains), and more preferably 20 or fewer.

Moreover, the term “alkyl” as used throughout the specification, examples, and claims is intended to include both unsubstituted and substituted alkyl groups, the latter of which refers to alkyl moieties having substituents replacing a hydrogen on one or more carbons of the hydrocarbon backbone, including haloalkyl groups such as trifluoromethyl and 2,2,2-trifluoroethyl, etc.

The term “C_{x-y}” or “Cₓ-C_{y}”, when used in conjunction with a chemical moiety, such as, acyl, acyloxy, alkyl, alkenyl, alkynyl, or alkoxy is meant to include groups that contain from x to y carbons in the chain. C₀alkyl indicates a hydrogen where the group is in a terminal position, a bond if internal. A C₁₋₆alkyl group, for example, contains from one to six carbon atoms in the chain.

The term “alkylamino”, as used herein, refers to an amino group substituted with at least one alkyl group.

The term “alkylthio”, as used herein, refers to a thiol group substituted with an alkyl group and may be represented by the general formula alkylS—.

The term “amido”, as used herein, refers to a group

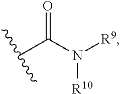

wherein R⁹ and R¹⁰ each independently represent a hydrogen or hydrocarbyl group, or R⁹ and R¹⁰ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

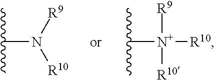

wherein R⁹, R¹⁰, and R¹⁰′ each independently represent a hydrogen or a hydrocarbyl group, or R⁹ and R¹⁰ taken together with the N atom to which they are attached complete a heterocycle having from 4 to 8 atoms in the ring structure.

The term “aminoalkyl”, as used herein, refers to an alkyl group substituted with an amino group.

The term “aralkyl”, as used herein, refers to an alkyl group substituted with an aryl group.

The term “aryl” as used herein include substituted or unsubstituted single-ring aromatic groups in which each atom of the ring is carbon. Preferably the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

The term “carbamate” is art-recognized and refers to a group

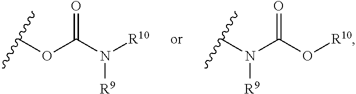

wherein R⁹ and R¹⁰ independently represent hydrogen or a hydrocarbyl group.

The term “carbocyclylalkyl”, as used herein, refers to an alkyl group substituted with a carbocycle group.

The term “carbocycle” includes 5-7 membered monocyclic and 8-12 membered bicyclic rings. Each ring of a bicyclic carbocycle may be selected from saturated, unsaturated and aromatic rings. Carbocycle includes bicyclic molecules in which one, two or three or more atoms are shared between the two rings. The term “fused carbocycle” refers to a bicyclic carbocycle in which each of the rings shares two adjacent atoms with the other ring. Each ring of a fused carbocycle may be selected from saturated, unsaturated and aromatic rings. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, is included in the definition of carbocyclic. Exemplary “carbocycles” include cyclopentane, cyclohexane, bicyclo [2.2.1]heptane, 1,5-cyclooctadiene, 1,2,3,4-tetrahydronaphthalene, bicyclo[4.2.0]oct-3-ene, naphthalene and adamantane. Exemplary fused carbocycles include decalin, naphthalene, 1,2,3,4-tetrahydronaphthalene, bicyclo [4.2.0]octane, 4,5,6,7-tetrahydro-1H-indene and bicyclo[4.1.0]hept-3-ene. “Carbocycles” may be substituted at any one or more positions capable of bearing a hydrogen atom.

The term “carbocyclylalkyl”, as used herein, refers to an alkyl group substituted with a carbocycle group.

The term “carbonate” is art-recognized and refers to a group —OCO₂—.

The term “carboxy”, as used herein, refers to a group represented by the formula —CO₂H.

The term “cycloalkyl” includes substituted or unsubstituted non-aromatic single ring structures, preferably 4- to 8-membered rings, more preferably 4- to 6-membered rings. The term “cycloalkyl” also includes polycyclic ring systems having two or more cyclic rings in which two or more carbons are common to two adjoining rings wherein at least one of the rings is cycloalkyl and the substituent (e.g., R¹⁰⁰) is attached to the cycloalkyl ring, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, pyrimidine, denzodioxane, tetrahydroquinoline, and the like.

The term “ester”, as used herein, refers to a group —C(O)OR⁹ wherein R⁹ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” as used herein means halogen and includes chloro, fluoro, bromo, and iodo.

The terms “hetaralkyl” and “heteroaralkyl”, as used herein, refers to an alkyl group substituted with a hetaryl group.

The terms “heteroaryl” and “hetaryl” include substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like. Accordingly, in certain embodiments, the present disclosure relates to derivatives and/or analogs of such exemplary heteroaryl systems as 1,3,4,5-tetrahydropyrrolo[4,3,2-de]quinoline and 3,4-dihydropyrrolo[4,3,2-de]quinolin-8(1H)-one.

The term “heteroatom” as used herein means an atom of any element other than carbon or hydrogen. Preferred heteroatoms are nitrogen, oxygen, and sulfur.

The term “heterocyclylalkyl”, as used herein, refers to an alkyl group substituted with a heterocycle group.

The terms “heterocyclyl”, “heterocycle”, and “heterocyclic” refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. The terms “heterocyclyl” and “heterocyclic” also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and even trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “lower” when used in conjunction with a chemical moiety, such as, acyl, acyloxy, alkyl, alkenyl, alkynyl, or alkoxy is meant to include groups where there are ten or fewer atoms in the substituent, preferably six or fewer. A “lower alkyl”, for example, refers to an alkyl group that contains ten or fewer carbon atoms, preferably six or fewer. In certain embodiments, acyl, acyloxy, alkyl, alkenyl, alkynyl, or alkoxy substituents defined herein are respectively lower acyl, lower acyloxy, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 5 to 7.

The term “sulfate” is art-recognized and refers to the group —OSO₃H, or a pharmaceutically acceptable salt thereof.

The term “sulfonamido” is art-recognized and refers to the group represented by the general formulae

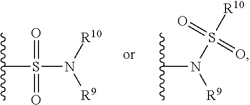

wherein R⁹ and R¹⁰ independently represents hydrogen or hydrocarbyl.

The term “sulfoxide” is art-recognized and refers to the group-S(O)—.

The term “sulfonate” is art-recognized and refers to the group SO₃H, or a pharmaceutically acceptable salt thereof.

The term “sulfone” is art-recognized and refers to the group —S(O)₂—.

The term “substituted” refers to moieties having substituents replacing a hydrogen on one or more carbons of the backbone. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, aphosphoryl, aphosphate, aphosphonate, aphosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

The term “thioalkyl”, as used herein, refers to an alkyl group substituted with a thiol group.

The term “thioester”, as used herein, refers to a group —C(O)SR⁹ or —SC(O)R⁹ wherein R⁹ represents a hydrocarbyl.

The term “thioether”, as used herein, is equivalent to an ether, wherein the oxygen is replaced with a sulfur.

The term “urea” is art-recognized and may be represented by the general formula

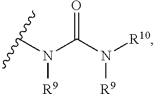

wherein R⁹ and R¹⁰ independently represent hydrogen or a hydrocarbyl.

The term “modulate” as used herein includes the inhibition or suppression of a function or activity (such as cell proliferation) as well as the enhancement of a function or activity.

The phrase “pharmaceutically acceptable” is art-recognized. In certain embodiments, the term includes compositions, excipients, adjuvants, polymers and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable salt” or “salt” is used herein to refer to an acid addition salt or a basic addition salt which is suitable for or compatible with the treatment of patients.

The term “pharmaceutically acceptable acid addition salt” as used herein means any non-toxic organic or inorganic salt of any base compounds represented by Formula I. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of compounds of Formula I are more soluble in water and various hydrophilic organic solvents, and generally demonstrate higher melting points in comparison to their free base forms. The selection of the appropriate salt will be known to one skilled in the art. Other non-pharmaceutically acceptable salts, e.g., oxalates, may be used, for example, in the isolation of compounds of Formula I for laboratory use, or for subsequent conversion to a pharmaceutically acceptable acid addition salt.

The term “pharmaceutically acceptable basic addition salt” as used herein means any non-toxic organic or inorganic base addition salt of any acid compounds represented by Formula I or any of their intermediates. Illustrative inorganic bases which form suitable salts include lithium, sodium, potassium, calcium, magnesium, or barium hydroxide. Illustrative organic bases which form suitable salts include aliphatic, alicyclic, or aromatic organic amines such as methylamine, trimethylamine and picoline or ammonia. The selection of the appropriate salt will be known to a person skilled in the art.

Many of the compounds useful in the methods and compositions of this disclosure have at least one stereogenic center in their structure. This stereogenic center may be present in a R or a S configuration, said R and S notation is used in correspondence with the rules described in Pure Appl. Chem. (1976), 45, 11-30. The disclosure contemplates all stereoisomeric forms such as enantiomeric and diastereoisomeric forms of the compounds, salts, prodrugs or mixtures thereof (including all possible mixtures of stereoisomers). See, e.g., WO 01/062726.

Furthermore, certain compounds which contain alkenyl groups may exist as Z (zusammen) or E (entgegen) isomers. In each instance, the disclosure includes both mixture and separate individual isomers.

Compounds of Formula I and sub-formulae thereof may exist in a number of different tautomeric forms and references to compounds of Formula I and sub-formulae thereof include all such forms. For the avoidance of doubt, where a compound can exist in one of several tautomeric forms, and only one is specifically described or shown, all others are nevertheless embraced by Formula I and sub-formulae thereof. Examples of tautomeric forms include keto-, enol-, and enolate-forms, as in, for example, the following tautomeric pairs: keto/enol (illustrated below, together with the deprotonated enolate salt form), 2-pyridone/2-hydroxypyridine, imine/enamine, amide/imino alcohol, amidine/enediamine, nitroso/oxime, thioketone/enethiol, and nitro/aci-nitro.

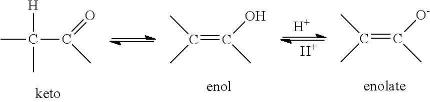

Compounds of Formula I and sub-formulae thereof may exist in oxidized or reduced forms, and references to compounds of Formula I and sub-formulae thereof include all such forms. For the avoidance of doubt, where a compound can exist in an oxidized or reduced form (e.g., without the loss/addition of non-hydrogen atoms), and only one is specifically described or shown, all others are nevertheless embraced by Formula I and sub-formula thereof. Examples of oxidized and reduced forms include quinone/hydroquinone and substituted 4-aminophenol/cyclohexadienoniminium. In certain such embodiments, compounds of Formula I and sub-formulae thereof may exist as the following oxidized and reduced forms:

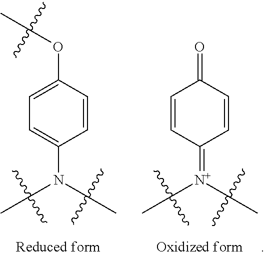 [SMILES: *Oc1ccc(N(*)*)cc1 ; *[N+](*)=C1C=CC(=O)C=C1]

“Prodrug” or “pharmaceutically acceptable prodrug” refers to a compound that is metabolized, for example hydrolyzed or oxidized, in the host after administration to form the compound of the present disclosure (e.g., compounds of formula I). Typical examples of prodrugs include compounds that have biologically labile or cleavable (protecting) groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, or dephosphorylated to produce the active compound. Examples of prodrugs using ester or phosphoramidate as biologically labile or cleavable (protecting) groups are disclosed in U.S. Pat. Nos. 6,875,751, 7,585,851, and 7,964,580, the disclosures of which are incorporated herein by reference. The prodrugs of this disclosure are metabolized to produce a compound of Formula I. The present disclosure includes within its scope, prodrugs of the compounds described herein. Conventional procedures for the selection and preparation of suitable prodrugs are described, for example, in “Design of Prodrugs” Ed. H. Bundgaard, Elsevier, 1985.

The phrase “pharmaceutically acceptable carrier” as used herein means a pharmaceutically acceptable material, composition or vehicle, such as a liquid or solid filter, diluent, excipient, solvent or encapsulating material useful for formulating a drug for medicinal or therapeutic use.

The term “Log of solubility”, “Log S” or “log S” as used herein is used in the art to quantify the aqueous solubility of a compound. The aqueous solubility of a compound significantly affects its absorption and distribution characteristics. A low solubility often goes along with a poor absorption. Log S value is a unit stripped logarithm (base 10) of the solubility measured in mol/liter.

### EXAMPLES

The invention now being generally described, it will be more readily understood by reference to the following examples which are included merely for purposes of illustration of certain aspects and embodiments of the present invention and are not intended to limit the invention.

### Example 1: Synthesis of Exemplary Compounds of the Disclosure

### Materials and Methods

Unless otherwise stated, reactions were performed in flame-dried glassware under an argon or nitrogen atmosphere using dry, deoxygenated solvents. Solvents were dried using known literature methods by passage through an activated alumina column under argon. NBS was recrystallized from boiling water prior to use, amines and TMSCl were distilled under nitrogen prior to use, and K₂CO₃ was flame-dried under vacuum and stored in a nitrogen-filled glovebox prior to use. All other reagents were purchased from commercial sources and used as received unless otherwise indicated. Reaction progress was monitored by thin-layer chromatography (TLC) or Agilent 1290 UHPLC-MS. TLC was performed using E. Merck silica gel 60 F254 precoated glass plates (0.25 mm) and visualized by UV fluorescence quenching or KMnO₄ staining. Silicycle SiliaFlash® P60 Academic Silica gel (particle size 40-63 nm) was used for flash chromatography. Preparative HPLC was performed on an Agilent 1100 Series HPLC system using a 9.4×250 mm Agilent Eclipse XDB-C18 column, or on an Agilent 1200 Series HPLC system using a 9.4×250 mm Agilent Zorbax Rx-SIL column. ¹H NMR spectra were recorded on Varian Inova 500 MHz, Varian 600 MHz, and Bruker 400 MHz spectrometers and are reported relative to residual CHC13 (δ 7.26 ppm), C₆D₆ (δ 7.16 ppm), DMSO-d₆ (δ 2.50 ppm), CD₂Cl₂ (δ 5.32 ppm), or CD₃OD (δ 3.31 ppm). ¹³C NMR spectra were recorded on a Bruker 400 MHz spectrometer (100 MHz) and are reported relative to CHCl₃ (δ 77.16 ppm), C₆D₆ (δ 128.06 ppm), DMSO-d₆ (δ 39.52 ppm), CD₂Cl₂ (δ 53.84 ppm), or CD₃OD (δ 49.00 ppm). Data for ¹H NMR are reported as follows: chemical shift (δ ppm) (multiplicity, coupling constant (Hz), integration). Multiplicities are reported as follows: s=singlet, d=doublet, t=triplet, q=quartet, p=pentet, sept=septuplet, m=multiplet, br s=broad singlet, br d=broad doublet. Data for ¹³C NMR are reported in terms of chemical shifts (δ ppm). Some reported spectra include minor solvent impurities of water, ethyl acetate, diethyl ether, methylene chloride, acetone, grease, and/or silicon grease, which do not impact product assignments. IR spectra were obtained by use of a Perkin Elmer Spectrum BXII spectrometer using thin films deposited on NaCl plates and reported in frequency of absorption (cm¹). High resolution mass spectra (HRMS) were obtained from an Agilent 6230 LC/TOF with an Agilent Jet Stream ion source in electrospray ionization (ESI+ or ESI−) mode, or from the Caltech Mass Spectrometry Laboratory using a JEOL JMS-T2000GC AccuTOF™ GC-Alpha in field desorption (FD+) mode.

### List of Abbreviations

CAN-Diammonium cerium(IV) nitrate, CDI-Carbonyldiimidazole, DDQ-2,3-Dichloro-5,6-dicyano-1,4-benzoquinone, DIAD-Diisopropyl azodicarboxylate, DIBAL-Diisobutylaluminium hydride, DMA-N,N-Dimethylacetamide, DMAP-N,N-Dimethylpyridin-4-amine, DPPA-Diphenoxyphosphoryl azide, HPLC-high-pressure liquid chromatography, LCMS-liquid chromatography/mass spectrometry, NBS—N—Bromosuccinimide, NMP-N-Methyl-2-pyrrolidone, NMR-nuclear magnetic resonance, TBSOTf-Trimethylsilyl trifluoromethanesulfonate, TFA-Trifluoroacetic acid, TFAA-Trifluoroacetic anhydride, THF-Tetrahydrofuran, TLC—thin layer chromatography, TsCl-4-Toluenesulfonyl chloride.

### EXPERIMENTAL PROCEDURES

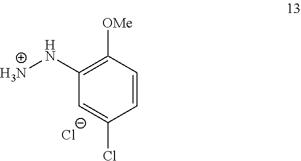 [SMILES: COc1ccc(Cl)cc1N[NH3+] ; [Cl-]]

### 2-(5-chloro-2-methoxyphenyl)hydrazin-1-ium chloride 13

Prepared according to the procedure of Thibeault and coworkers from 4-chloro-2-methoxyaniline.² All characterization data matched those reported in the literature.

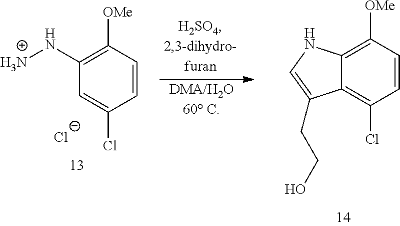 [SMILES: COc1ccc(Cl)cc1N[NH3+] ; [Cl-] ; COc1ccc(Cl)c2c(CCO)c[nH]c12]

### 2-(4-chloro-7-methoxy-1H-indol-3-yl)ethan-1-ol 14

To a 250 mL round bottom flask were added (5-chloro-2-methoxyphenyl)hydrazine hydrochloride (13, 8.60 g, 41.1 mmol, 1.0 equiv). DMA (65 mL), H₂O (65 mL), and 25% (v/v) aq. H₂SO₄ (11.0 mL, 51.4 mmol, 1.25 equiv). 2,3-dihydrofuran (3.42 mL, 45.2 mmol, 1.1 equiv) was added slowly with stirring, resulting in a slow color change to yellow, whereafter the flask was equipped with an air-cooled reflux condenser and heated to 60° C. in an oil bath for 6 h. The reaction mixture was then allowed to cool to 23° C. and transferred to a separatory funnel containing 1:1 saturated aq. NaHCO₃/brine (250 mL). The mixture was then extracted with EtOAc (4×100 mL), and the combined organic extracts were washed with water, dried over Na₂SO₄, and concentrated under reduced pressure. The crude product was purified by silica gel flash chromatography (60% EtOAc/hexanes) to afford the title compound as a thick, red-brown oil that slowly crystallized upon storage at −20° C. (2.66 g, 11.79 mmol, 29% yield).

¹H NMR (400 MHz, CDCl₃): δ 8.54 (br s, 1H), 7.00 (d, J=2.2 Hz, 1H), 6.96 (d, J=8.2 Hz, 1H), 6.50 (d, J=8.2 Hz, 1H), 3.94 (t, J=6.4 Hz, 2H), 3.91 (s, 3H), 3.23 (td, J=6.4, 0.8 Hz, 2H)

¹³C NMR (100 MHz, CDCl₃): δ 145.2, 128.4, 124.8, 123.8, 120.2, 118.2, 113.1, 102.4, 63.7, 55.7, 29.5.

IR (Neat Film, NaCl): 3416, 2935, 2580, 1786, 1703, 1626, 1571, 1494, 1452, 1339, 1230, 1115, 1052, 937, 789, 736, 694, 628 cm⁻¹.

HRMS (FD+): m/z calc'd for C₁₁H₁₃ClNO₂ [M+H]⁺: 225.0557, found 225.0552.

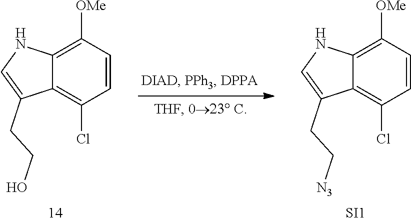

### 3-(2-azidoethyl)-4-chloro-7-methoxy-1H-indole SI1

To a 250 mL round bottom flask was added tryptophol 14 (3.36 g, 14.89 mmol, 1.0 equiv). Traces of water were azeotropically removed by addition of three portions of benzene followed by rotary evaporation. PPh₃ (4.69 g, 17.87 mmol, 1.2 equiv) was added, and the headspace of the flask was evacuated and backfilled with nitrogen. THF (60 mL) was added, and the reaction mixture was cooled to 0° C. Then, DIAD (3.52 mL, 17.87 mmol, 1.2 equiv) was added dropwise over 4 min. The reaction mixture was stirred at 0° C. for an additional 15 min, after which DPPA (3.85 mL, 17.87 mmol, 1.2 equiv) was added rapidly and the reaction mixture was allowed to warm to 23° C. The reaction mixture became cloudy over several minutes. TLC analysis after 16 h indicated a high degree of conversion to product. Stirring for another 24 h led to no change in the reaction profile by TLC analysis. The reaction mixture was concentrated under reduced pressure and purified by automated silica gel flash chromatography (Teledyne ISCO, 0→40% EtOAc/hexanes) to afford azide SI1 as an orange-brown solid (2.50 g, 9.97 mmol, 67% yield).

¹H NMR (600 MHz, CDCl₃): δ 8.34 (s, 1H), 7.05 (d, J=2.4 Hz, 1H), 6.98 (d, J=8.2 Hz, 1H), 6.53 (d, J=8.2 Hz, 1H), 3.93 (s, 3H), 3.59 (t, J=7.2 Hz, 2H), 3.27 (td, J=7.1, 0.8 Hz, 2H).

¹³C NMR (100 MHz, CDCl₃): δ 145.2, 128.2, 124.5, 123.5, 120.3, 117.9, 113.2, 102.5, 55.6, 52.8, 25.9.

IR (Neat Film, NaCl): 3890, 3864, 3668, 3316, 3132, 3084, 3008, 2938, 2876, 2588, 2492, 2352, 2204, 2106, 1768, 1652, 1630, 1574, 1494, 1464, 1450, 1434, 1380, 1332, 1294, 1272, 1236, 1194, 1172, 1154, 1130, 1070, 1034, 958, 930, 864, 830, 808, 782, 760, 740, 708, 684, 670, 662, 632, cm¹.

HRMS (ESI+): m/z calc'd for C₁₁H₁₂ClN₂O [M+H]⁺: 223.0638, found 223.0630.

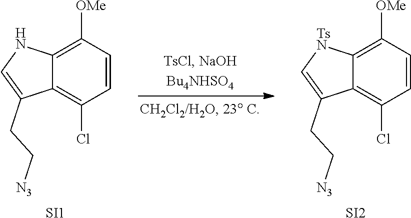

### Tosyl Indole SI2

To a 250 mL round bottom flask under air were added azide SI1 (5.11 g, 20.4 mmol, 1.0 equiv), CH₂Cl₂ (41 mL), and Bu₄NHSO₄ (69 mg, 0.204 mmol, 1 mol %), followed by 50% w/v aq. NaOH (14 mL). After stirring for 5 min, TsCl (7.78 g, 40.8 mmol, 2.0 equiv) was added in a single portion. The reaction mixture was subjected to vigorous magnetic stirring for 10 min, then diluted with H₂O (100 mL) and CH₂Cl₂ (30 mL). The layers were separated, and the aqueous phase was extracted with CH₂Cl₂ (3×50 mL). The combined organic phases were washed with brine, dried over Na₂SO₄, and concentrated under reduced pressure. The crude product was purified by automated silica gel flash chromatography (Teledyne ISCO, 0->45% EtOAc/hexanes) to afford an intermediate tosyl indole as a white solid (6.77 g, 16.7 mmol, 82% yield).

¹H NMR (400 MHz, CDCl₃): δ 7.74 (s, 1H), 7.71 (d, J=8.4 Hz, 2H), 7.29-7.26 (m, 2H), 7.08 (d, J=8.5 Hz, 1H), 6.58 (d, J=8.5 Hz, 1H), 3.64 (s, 3H), 3.63-3.59 (m, 2H), 3.26 (td, J=7.1, 1.0 Hz, 2H), 2.40 (s, 3H).

¹³C NMR (100 MHz, CDCl₃): δ 146.9, 144.9, 137.6, 129.9, 129.6, 128.6, 127.6, 126.7, 125.0, 118.5, 116.7, 108.0, 56.1, 52.2, 26.7, 22.1.

IR (Neat Film, NaCl): 3992, 3892, 3854, 3752, 3630, 3620, 3608, 3586, 3402, 3116, 2926, 2514, 2368, 2098, 1598, 1572, 1540, 1518, 1488, 1464, 1362, 1294, 1252, 1234, 1192, 1172, 1146, 1120, 1092, 1058, 994, 910, 858, 846, 808, 798, 772, 736, 702, 676, 662, 632, 624, cm⁻¹
HRMS (ESI+): m/z calc'd for C₁₈H₁₇ClN₄O₃SNa [M+Na]⁺: 427.0608, found 427.0603.

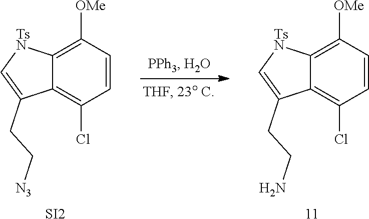

### Tryptamine 11

To a 500 mL round bottom flask under air were added this tosyl azide SI2 (11.73 g, 28.97 mmol, 1.0 equiv), PPh₃ (9.88 g, 37.66 mmol, 1.3 equiv), and THF (190 mL). The reaction mixture was stirred at 23° C. for 15 h, whereafter deionized water (9.5 mL) was added. After an additional 30 h, the reaction mixture was concentrated under reduced pressure and purified by silica gel flash chromatography (10% MeOH/CH₂Cl₂+1% Et₃N) to afford the title compound as a white solid (10.41 g, 27.48 mmol, 95% yield)

¹H NMR (400 MHz, CDCl₃): δ 7.72-7.65 (m, 3H), 7.29-7.23 (m, 2H), 7.05 (d, J=8.5 Hz, 1H), 6.56 (d, J=8.5 Hz, 1H), 3.62 (s, 3H), 3.14-3.00 (m, 4H), 2.39 (s, 3H).

¹³C NMR (100 MHz, CDCl₃): δ 146.5, 144.4, 137.4, 129.7, 129.5, 127.4, 127.2, 126.5, 124.6, 118.5, 118.1, 107.5, 55.8, 42.9, 30.7, 21.7.

IR (Neat Film, NaCl): 2938, 2686, 2595, 2515, 2363, 1574, 1487, 1366, 1290, 1237, 1171, 1092, 1052, 997, 937, 809, 662 cm⁻¹.

HRMS (ESI+): m/z calc'd for C₁₈H₂OClN₂O₃S [M+H]⁺: 379.0878, found 379.0877.

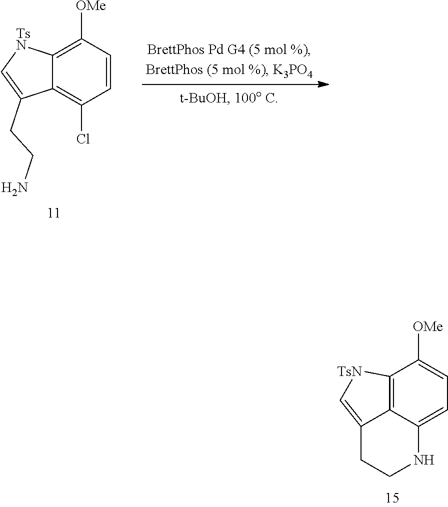

### Tricycle 15

A 20 mL glass vial containing tryptamine 11 (1.0 g, 2.64 mmol, 1.0 equiv) was brought into a nitrogen-filled glovebox. To this vial were added BrettPhos Pd G4 (121 mg, 0.132 mmol, 5 mol %), BrettPhos (71 mg, 0.132 mmol, 5 mol %), and K₃PO₄ (784 mg, 3.70 mmol, 1.4 equiv), followed by t-BuOH (6.6 mL). The vial was sealed with a PTFE-lined cap and electrical tape, removed from the glovebox, and stirred at 100° C. in a metal heating block. After 3 days, the reaction mixture was partitioned between CH₂Cl₂ and water and the layers were separated. The aqueous phase was extracted with CH₂Cl₂ (3×), and the combined organic phases were concentrated under reduced pressure. The crude product was purified by silica gel flash chromatography (40% EtOAc/hexanes) to afford the title compound as a beige foam (816 mg, 2.38 mmol, 90% yield).

¹H NMR (400 MHz, CDCl₃): δ 7.79 (d, J=8.4 Hz, 2H), 7.32 (s, 1H), 7.23 (d, J=7.9 Hz, 2H), 6.56 (d, J=8.1 Hz, 1H), 6.27 (d, J=8.1 Hz, 1H), 3.70 (s, 3H), 3.37 (t, J=5.9 Hz, 2H), 2.91 (t, J=5.2 Hz, 2H), 2.36 (s, 3H).

¹³C NMR (100 MHz, CDCl₃): δ 144.1, 140.5, 137.1, 135.8, 129.4, 127.7, 124.0, 122.6, 120.2, 115.2, 110.7, 105.0, 57.5, 43.0, 22.8, 21.7.

IR (Neat Film, NaCl): 3384, 2956, 2834, 1595, 1512, 1421, 1356, 1260, 1170, 1103, 1035, 977, 937, 795, 664 cm¹.

HRMS (ESI+): m/z calc'd for C₁₈H₁₉N₂O₃S [M+H]⁺: 343.1111, found 343.1127.

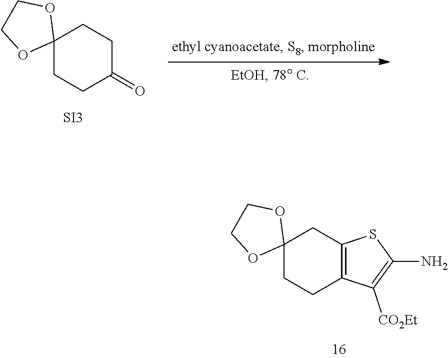

### Aminothiophene 16

Prepared according to known literature methods from 1,4-cyclohexandione monoethylene ketal (SI3). To a 500 mL round bottom flask were added 1,4-cyclohexanedione monoethylene ketal (20 g, 128 mmol, 1.0 equiv), S₈ (4.1 g, 128 mmol, 1.0 equiv), ethyl cyanoacetate (14 mL, 128 mmol, 1.0 equiv), morpholine (11 mL, 128 mmol, 1.0 equiv), and EtOH (250 mL). The mixture was refluxed for 12 h at which point TLC analysis showed full conversion. The reaction mixture was cooled to room temperature and concentrated under reduced pressure until the amount of solution became approximately half. Then the product was collected by vaccum filtration to afford aminothiophene 16 as a light yellow solid (30.3 g, 107 mmol, 83% yield). All characterization data matched those reported in the literature.

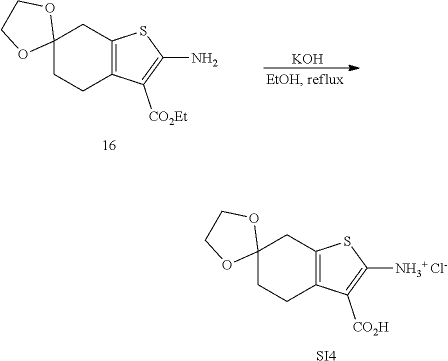

### Amino Acid Hydrochloride SI4

To a 350 mL glass pressure vessel under air were added aminothiophene 16 (22.2 g, 78.35 mmol, 1.0 equiv), KOH (17.6 g, 313.4 mmol, 4.0 equiv), EtOH (165 mL), and water (33 mL). The flask was sealed, and the reaction mixture was stirred at 75° C. in an oil bath for 12 h. The reaction mixture was subsequently transferred to an Erlenmeyer flask and cooled to 0° C. in an ice bath. The solution was acidified with 1 N aq. HCl (375 mL), resulting in the formation of a thick precipitate. The product was collected by vacuum filtration and washed with water. The resulting clay-like material was transferred to a round bottom flask with MeOH. The solvent was removed under reduced pressure and the product was dried under high vacuum to afford the title compound as a cream-colored powder (17.95 g, 61.53 mmol, 79% yield assuming complete conversion to hydrochloride). Note: Conducting this transformation in a vessel with increased headspace generally leads to lower yields, likely due to atmospheric oxidation.

¹H NMR (400 MHz, DMSO): δ 11.81 (br s, 1H), 7.20 (br s, 2H), 3.90 (s, 4H), 2.72 (t, J=6.4 Hz, 2H), 2.59 (s, 2H), 1.75 (t, J=6.5 Hz, 2H).

¹³C NMR (100 MHz, DMSO): δ 166.7, 163.4, 130.9, 112.4, 107.6, 102.6, 63.9, 34.3, 31.0, 25.1.

IR (Neat Film, NaCl): 3409, 2890, 1635, 1580, 1474, 1451, 1353, 1294, 1267, 1105, 1054, 1036, 926, 642 cm¹.

HRMS (ESI−): m/z calc'd for C_{II}H₁₂NO₄S [M−H]⁻: 254.0493, found 254.0494.

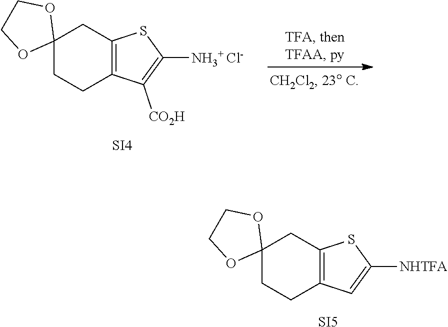

### Trifluoroacetamidothiophene SI5

To a 500 mL round bottom flask was added hydrochloride salt SI4 (14.29 g, 48.98 mmol, 1.0 equiv). Then, the headspace of the flask was evacuated and backfilled with nitrogen, and CH₂Cl₂ (150 mL) and TFA (11.2 mL, 146.9 mmol, 3.0 equiv) were added. After 15 min, additional CH₂Cl₂ (50 mL) was added to reduce the viscosity of the mixture. The reaction mixture was stirred for 2.5 h at 23° C., whereafter it was cooled to 0° C. in an ice bath and pyridine (19.7 mL, 244.9 mmol, 5.0 equiv) was added rapidly. TFAA (10.2 mL, 73.47 mmol, 1.5 equiv) was then added, and the ice bath was removed. After stirring for an additional 16 h at 23° C., silica gel was added, and the reaction mixture was concentrated under reduced pressure and purified by silica gel flash chromatography (33% EtOAc/hexanes) to afford the title compound as an orange solid (9.67 g, 31.47 mmol, 64% yield).

¹H NMR (400 MHz, CDCl₃): δ 8.72 (s, 1H), 6.59 (s, 1H), 4.06-3.98 (m, 4H), 2.91 (d, J=1.5 Hz, 2H), 2.74 (tt, J=6.5, 1.6 Hz, 2H), 1.94 (t, J=6.5 Hz, 2H).

¹³C NMR (100 MHz, CDCl₃): δ 153.6 (q, J=38.3 Hz), 133.4, 131.8, 127.9, 117.0 (q, J=287.1 Hz), 116.1, 108.5, 64.8, 34.9, 31.7, 24.0.

IR (Neat Film, NaCl): 3248, 3097, 2894, 1713, 1589, 1434, 1362, 1250, 1169, 1060, 946, 905, 842, 739 cm⁻¹.

HRMS (ESI+): m/z calc'd for Cl₂H₁₃F₃NO₃S [M+H]⁺: 308.0563, found 308.0561.

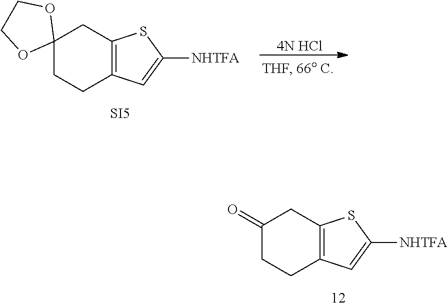

### Thiophene-Ketone 12

To a 100 mL round bottom flask containing thiophene-ketal SI5 (1.64 g, 5.34 mmol, 1.5 equiv) under nitrogen and equipped with a reflux condenser were added THF (27 mL) and 4 N aq. HCl (5.3 mL, 21.2 mmol, 4.0 equiv w.r.t. ketal). The reaction mixture was heated to reflux and stirred for 1.5 h, then allowed to cool and quenched with saturated aq. NaHCO₃ (100 mL). The resulting mixture was extracted with CH₂Cl₂ (3×40 mL). The combined organic extracts were washed with brine, and the brine phase was back-extracted once with CH₂Cl₂. The combined organic phases were dried over Na₂SO₄ and MgSO4 and concentrated under reduced pressure to afford crude thiophene-ketone 12 as an orange solid that was used directly without further purification.

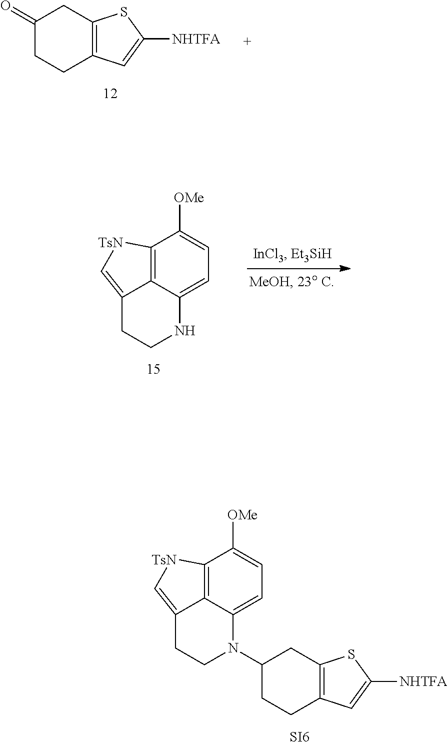

### Arene SI6

To a 50 mL round bottom flask were added tricyclic aniline 15 (1.22 g, 3.56 mmol, 1.0 equiv), the crude thiophene-ketone resulting from hydrolysis of trifluoroacetamidothiophene SI5 (th. 5.34 mmol), and InCl₃ (1.57 g, 7.12 mmol, 2.0 equiv). The headspace of the flask was evacuated and replaced with nitrogen. MeOH (12 mL) and Et₃SiH (5.7 mL, 35.6 mmol, 10 equiv) were added and the reaction mixture was stirred at 23° C. for 24 h, whereafter it was transferred to a separatory funnel containing saturated aq. NaHCO₃ (200 mL). The resulting suspension was extracted with CH₂Cl₂ (3×70 mL). The combined organic extracts (still containing a significant quantity of water and indium salts) were dried over Na₂SO₄, concentrated under reduced pressure, and purified by automated silica gel flash chromatography (Teledyne ISCO, 0→60% EtOAc/hexanes) to afford the title compound as a golden-colored foam of sufficient purity for use in the next step (1.80 g, 3.05 mmol, 86% yield). A sample for analysis was obtained by preparative TLC on silica gel (33% EtOAc/hexanes).

¹H NMR (400 MHz, CDCl₃): δ 8.40 (br s, 1H), 7.82-7.75 (m, 2H), 7.34 (t, J=1.5 Hz, 1H), 7.26-7.21 (m, 2H), 6.60 (s, 1H), 6.58 (d, J=8.3 Hz, 1H), 6.30 (d, J=8.8 Hz, 1H), 4.15-4.01 (m, 1H), 3.68 (s, 3H), 3.33 (dt, J=11.0, 5.4 Hz, 1H), 3.21 (dt, J=11.5, 6.2 Hz, 1H), 2.99-2.92 (m, 3H), 2.91-2.66 (m, 3H), 2.37 (s, 3H), 2.07-1.98 (m, 2H).

¹³C NMR (100 MHz, CDCl₃): δ 153.6 (q, J=38.1 Hz), 144.2, 140.0, 137.1, 136.3, 133.2, 132.4, 129.5, 129.4, 127.7, 124.2, 123.2, 119.7, 116.5, 115.4, 114.4 (q, J=287.3 Hz), 110.3, 102.7, 57.3, 53.5, 42.2, 27.0, 26.0, 25.9, 23.5, 21.7.

IR (Neat Film, NaCl): 3281, 3126, 3058, 2935, 2841, 1714, 1588, 1510, 1442, 1356, 1251, 1169, 1108, 1056, 996, 903, 739, 663 cm⁻¹.

HRMS (ESI+): m/z calc'd for C₂₈H₂₇F3N₃O₄S₂ [M+H]⁺: 590.1390, found 590.1404.

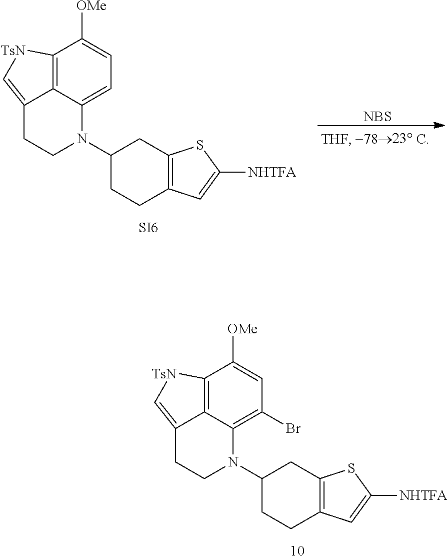

To a 250 mL round bottom flask was added arene SI6 (1.78 g, 3.02 mmol, 1.0 equiv) and the headspace of the flask was evacuated and backfilled with nitrogen. The starting material was taken up in THF (50 mL), and the flask was cooled to −78° C. and protected from light. A solution of NBS (484 mg, 2.72 mmol, 0.9 equiv) in THF (50 mL) was added as a slow stream with rapid stirring. Following addition, the reaction mixture was allowed to stir at −78° C. for 5 additional minutes, whereafter the cooling bath was removed and the reaction mixture allowed to warm to 23° C. The reaction mixture was concentrated under reduced pressure and purified by automated silica gel flash chromatography (Teledyne ISCO, 0→50% EtOAc/hexanes) to afford the title compound as a brown foam (1.16 g, 1.74 mmol, 57% yield from arene SI6 or 64% from NBS).

¹H NMR (400 MHz, C₆D₆): δ 7.83 (d, J=8.3 Hz, 2H), 7.54 (s, 1H), 6.75 (s, 1H), 6.64 (d, J=8.2 Hz, 2H), 6.01 (s, 1H), 4.17 (dddd, J=13.7, 11.3, 5.3, 2.6 Hz, 1H), 3.19 (s, 3H), 2.99-2.75 (m, 3H), 2.58 (tdd, J=11.7, 3.9, 2.0 Hz, 1H), 2.36-2.09 (m, 4H), 1.80-1.71 (m, 1H), 1.76 (s, 3H), 1.45 (tdd, J=12.2, 10.7, 6.7 Hz, 1H).

¹³C NMR (100 MHz, C₆D₆): δ 153.2 (q, J=38.7 Hz), 144.1, 143.0, 137.8, 134.6, 133.6, 131.9, 129.6, 129.5, 123.1, 122.4, 116.6, 115.2, 114.2, 106.2, 59.5, 56.3, 43.0, 29.5, 28.5, 26.0, 22.6, 21.1. (Note: the —CF₃ carbon is omitted due to low intensity resulting from C—F splitting. Two of the tosyl signals are obscured by the solvent peak.)

IR (Neat Film, NaCl): 3295, 2931, 1714, 1588, 1490, 1438, 1347, 1251, 1172, 1111, 908, 804, 737, 665 cm¹.

HRMS (ESI+): m/z calc'd for C₂₈H₂₆BrF₃N₃O₄S₂[M+H]⁺: 668.0495, found 668.0502.

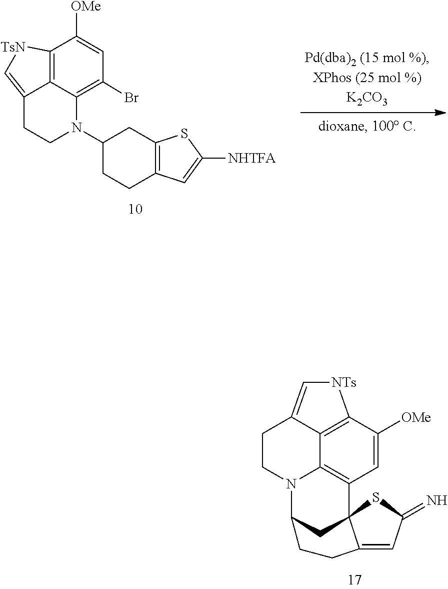

### Thioimidate 17

To a 40 mL glass vial was added bromoarene 10 (331 mg, 0.495 mmol, 1.0 equiv). Traces of water were azeotropically removed by addition of three portions of benzene followed by rotary evaporation, the vial was transferred to a nitrogen-filled glovebox, and K₂CO₃ (96 mg, 0.695 mmol, 1.4 equiv) was added. In a separate vial, a stock solution of Pd(dba)₂ (46.2 mg) and XPhos (64.2 mg) in 1,4-dioxane (13.3 mL) was prepared and stirred at 28° C. for 10 min. Then, 12.4 mL of this solution were transferred to the vial containing bromoarene 10 (for 43 mg Pd(dba)₂ (0.0748 mmol, 15 mol %) and 59.1 mg XPhos (0.124 mmol, 25 mol %)). The vial was sealed with a PTFE/silicone septum cap and electrical tape, removed from the glovebox, and stirred at 100° C. for 6 h. The reaction mixture was then allowed to cool and passed through a PTFE syringe filter, which was subsequently rinsed with EtOAc. The solution was concentrated under reduced pressure and purified by silica gel flash chromatography (100% EtOAc→100% acetone) to afford the title compound as an off-white solid (145 mg, 0.295 mmol, 60% yield).

¹H NMR (400 MHz, CD₂Cl₂): δ 7.81-7.72 (m, 2H), 7.32-7.24 (m, 3H), 6.81 (s, 1H), 5.78 (d, J=1.7 Hz, 1H), 3.64 (s, 3H), 3.59 (p, J=2.7 Hz, 1H), 3.35 (ddd, J=11.1, 9.7, 5.7 Hz, 1H), 3.25 (ddd, J=11.1, 4.8, 3.5 Hz, 1H), 3.04-2.94 (m, 2H), 2.65 (dt, J=12.5, 2.9 Hz, 1H), 2.54-2.47 (m, 1H), 2.40 (qd, J=5.3, 3.1 Hz, 1H), 2.37 (s, 3H), 2.33 (dd, J=12.5, 2.9 Hz, 1H), 2.06-1.97 (m, 1H), 1.61 (tdd, J=13.7, 5.3, 2.9 Hz, 1H).

¹³C NMR (100 MHz, CD₂Cl₂): δ 178.5, 168.3, 144.9, 139.3, 137.2, 133.5, 129.7, 128.0, 123.5, 122.3, 121.7, 120.6, 115.2, 114.5, 109.1, 63.5, 57.4, 47.6, 41.9, 31.9, 24.8, 23.2, 21.7 (the bridgehead methine ¹³C resonance is obscured by the solvent peak).

IR (Neat Film, NaCl): 2925, 2851, 1595, 1503, 1462, 1349, 1284, 1226, 1174, 1106, 1011, 898, 808, 738, 704, 672 cm.

HRMS (ESI+): m/z calc'd for C₂₆H₂₆N₃O₃S₂[M+H]⁺: 492.1410, found 492.1410.

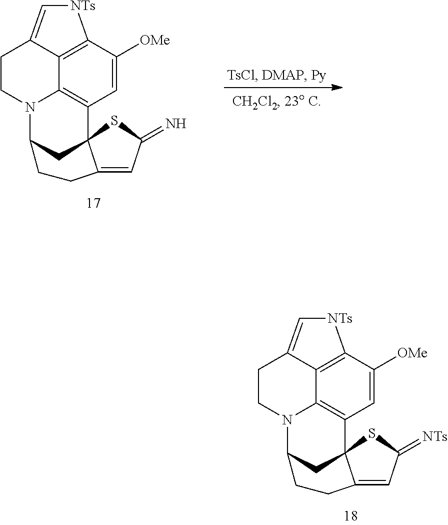

### N-tosylthioimidate 18

To a 1-dram glass vial containing TsCl (16 mg, 0.0814 mmol, 2.0 equiv) under nitrogen was added thioimidate 17 (20 mg, 0.0407 mmol, 1.0 equiv) as a stock solution in CH₂Cl₂ (1 mL), followed by pyridine (10 μL, 0.122 mmol, 3.0 equiv). The reaction mixture was stirred at 23° C. for 3 days, after which it was concentrated under reduced pressure and purified by silica gel flash chromatography (50% EtOAc/hexanes) to afford the title compound as an orange film (23.7 mg, 0.0367 mmol, 90% yield).

¹H NMR (400 MHz, CDCl₃): δ 7.93 (d, J=8.1 Hz, 2H), 7.83-7.74 (m, 2H), 7.36-7.28 (m, 3H), 7.28-7.22 (m, 2H), 6.49 (s, 1H), 5.97 (d, J=1.6 Hz, 1H), 3.65-3.61 (m, 1H), 3.59 (s, 3H), 3.37 (dt, J=10.9, 7.6 Hz, 1H), 3.27 (dt, J=11.1, 4.2 Hz, 1H), 3.06-2.97 (m, 2H), 2.83 (dt, J=12.6, 2.8 Hz, 1H), 2.68-2.58 (m, 1H), 2.49-2.44 (m, 1H), 2.43 (s, 3H), 2.38 (s, 3H), 2.21-2.04 (m, 2H), 1.59-1.51 (m, 1H).

¹³C NMR (100 MHz, CDCl₃): δ 184.5, 175.3, 144.4, 144.0, 139.4, 137.3, 136.9, 133.1, 129.6, 129.5, 127.9, 127.6, 123.6, 122.7, 121.6, 120.7, 114.2, 111.1, 107.8, 65.4, 57.2, 53.0, 47.4, 40.4, 31.7, 25.4, 23.0, 21.7. (Note: The two tosyl —CH₃ peaks overlap.) IR (Neat Film, NaCl): 2924, 2850, 1596, 1519, 1449, 1286, 1228, 1167, 1107, 1010, 867, 813, 734, 664 cm¹.

HRMS (ESI+): m/z calc'd for C₃₃H₃₂N₃O₅S₃[M+H]⁺: 646.1499, found 646.1500.

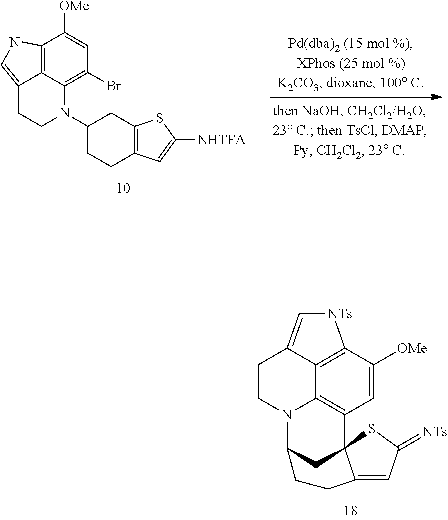

### Procedure for the Direct Preparation of N-Tosylthioimidate 18

To a 500 mL Schlenk bomb in a nitrogen-filled glovebox was added bromoarene 10 (3.88 g, 5.80 mmol, 1.0 equiv) as a solution in a minimal amount of benzene. The benzene was removed under reduced pressure, and K₂CO₃ (1.12 g, 8.12 mmol, 1.4 equiv) and 1,4-dioxane (110 mL) were added. In a separate vial, Pd(dba)₂ (500 mg, 0.870 mmol, 15 mol %) and XPhos (691 mg, 1.45 mmol, 25 mol %) were combined. 1,4-dioxane (35 mL) was added and the mixture was stirred at 28° C. for 10 min. Then, the Pd/ligand solution was transferred by pipette to the Schlenk bomb. The vessel was sealed, removed from the glovebox, and stirred at 100° C. for 7 h. The reaction mixture was allowed to cool, filtered through a Celite plug with EtOAc, and concentrated under reduced pressure.

The residue was taken up in CH₂Cl₂ (70 mL). 1 N aq. NaOH was added, and the biphasic mixture was subjected to vigorous magnetic stirring for 1 h. The layers were separated, and the aqueous phase was extracted with CH₂Cl₂ (3×20 mL). The combined organic phases were dried over Na₂SO₄ and concentrated under reduced pressure in a 500 mL round bottom flask.

Traces of water were azeotropically removed by addition of three portions of PhCH₃ followed by rotary evaporation. DMAP (71 mg, 0.58 mmol, 10 mol %) was added and the headspace of the flask was evacuated and replaced with nitrogen. CH₂Cl₂ (115 mL) was added, followed by pyridine (1.4 mL, 17.4 mmol, 3.0 equiv). TsCl (2.21 g, 11.6 mmol, 2.0 equiv) was added in a single portion under a stream of nitrogen. The reaction mixture was stirred at 23° C. for 58 h, then concentrated under reduced pressure. The crude product was purified by automated silica gel flash chromatography (Teledyne ISCO, 0→40→75% EtOAc/hexanes) to afford N-tosylthioimidate 18 as an orange foam that crystallized upon standing (2.66 g, 4.12 mmol, 71% yield). For characterization data, see above.

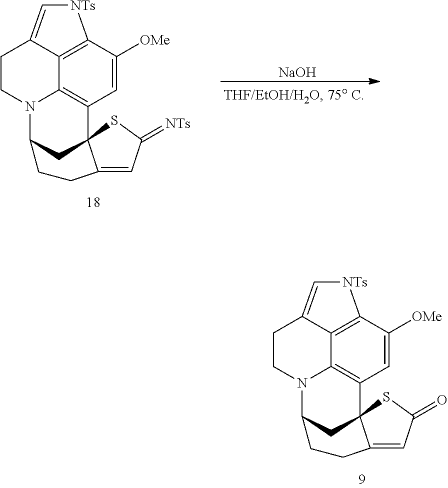

### Thiobutenolide 9

To a 250 mL round bottom flask were added thioimidate 18 (940 mg, 1.46 mmol, 1.0 equiv), THF (30 mL), water (10 mL), degassed ethanol (50 mL), and 2 N aq. NaOH (6 mL, 12 mmol, 8.2 equiv). The headspace of the vial was purged with nitrogen and the vial was sealed with a PTFE/silicone septum. The reaction mixture was stirred at 80° C. for 1 hour, at which point the solution became homogenous and was immediately removed from the oil bath and allowed to cool to 23° C. The reaction mixture was quenched with glacial acetic acid (2 mL) and partitioned between water and EtOAc. The layers were separated, and the aqueous phase was extracted with EtOAc (3×). The combined organic phases were dried over Na₂SO₄ and concentrated in vacuo to afford the crude product. Purification by silica gel flash chromatography (0->2% EtOAc/CH₂Cl₂) afforded the title compound (591 mg, 1.21 mmol, 83% yield).

¹H NMR (400 MHz, C₆D₆): δ 7.86 (d, J=8.2 Hz, 2H), 7.47 (d, J=1.6 Hz, 1H), 6.89 (t, J=1.3 Hz, 1H), 6.67-6.59 (m, 2H), 5.50 (d, J=1.7 Hz, 1H), 3.32 (s, 3H), 2.87-2.78 (m, 1H), 2.78-2.59 (m, 2H), 2.59-2.45 (m, 2H), 2.40 (dt, J=12.4, 2.8 Hz, 1H), 1.91-1.81 (m, 1H), 1.81-1.76 (m, 1H), 1.74 (s, 3H), 1.73-1.66 (m, 1H), 1.66-1.56 (m, 1H), 0.97-0.81 (m, 1H).

¹³C NMR (100 MHz, C₆D₆): δ 196.3, 177.3, 143.9, 140.0, 137.8, 133.5, 129.4, 124.2, 121.9, 121.7, 121.0, 114.6, 112.8, 108.9, 60.8, 57.0, 53.0, 47.1, 40.9, 31.4, 24.8, 23.0, 21.1 (Note: an additional ¹³C resonance associated with the tosyl group is likely obscured by the solvent signal).

IR (Neat Film, NaCl): 2931, 2851, 1682, 1504, 1348, 1284, 1227, 1169, 1107, 1011, 897, 851, 814, 738, 662 cm¹.

HRMS (ESI+): m/z calc'd for C₂₆H₂₅N₂O₄S₂[M+H]⁺: 493.1250, found 493.1251.

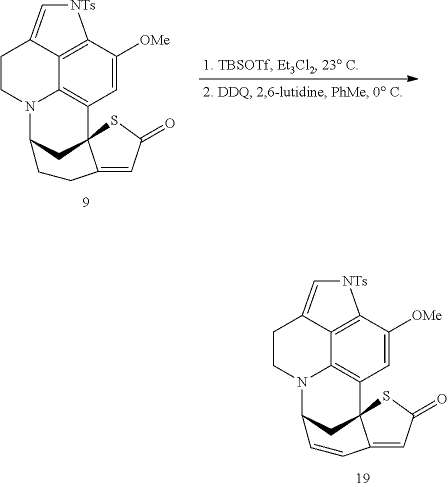 [SMILES: COc1cc2c3c4c(cn(S(=O)(=O)c5ccc(C)cc5)c14)CCN3[C@H]1CCC3=CC(=O)S[C@]32C1 ; COc1cc2c3c4c(cn(S(=O)(=O)c5ccc(C)cc5)c14)CCN3[C@H]1C=CC3=CC(=O)S[C@]32C1]

### Diene 19

To a 20 mL glass vial containing thiobutenolide 9 (50 mg, 0.102 mmol, 1.0 equiv) under nitrogen was added CH₂Cl₂ (1 mL), triethylamine (0.15 mL, 1.02 mmol, 10 equiv), and TBSOTf (0.16 mL, 0.711 mmol, 7.0 equiv). The reaction mixture was stirred at 23° C. for 14 hours before being loaded on to a silica gel column. The column was flushed with 10% EtOAc/hexanes (200 mL), followed by 40% EtOAc/hexanes (150 mL) to afford an intermediate silyl ketene thioacetal and starting material (12.7 mg, 0.0258 mmol, 25% recovery). The silyl ketene thioacetal was transferred to an 8 mL vial, evacuated and backfilled 3 times with N₂, and PhMe (0.76 mL) was added. The resulting mixture was cooled to 0° C. in an ice bath, whereafter 2,6-lutidine (17.5 μL, 0.152 mmol, 2.0 equiv) and DDQ (17.3 mg, 0.0761 mmol, 1.0 equiv, as a solution in 0.76 mL PhMe) were sequentially added. The reaction mixture was stirred at 0° C. and the reaction was monitored by LC/MS. Upon the completion of the reaction (ca. ˜5 min), the reaction mixture was loaded directly onto silica gel. Purification by silica gel flash chromatography (0→35% EtOAc/hexanes) afforded diene 19 (26.3 mg, 0.0537 mmol, 53% over 2 steps) as an orange solid.

¹H NMR (400 MHz, C₆D₆): δ 7.83-7.78 (m, 2H), 7.44 (d, J=1.6 Hz, 1H), 6.92 (s, 1H), 6.62-6.53 (m, 2H), 5.87 (ddd, J=9.7, 1.9, 0.9 Hz, 1H), 5.52 (s, 1H), 5.46-5.38 (m, 1H), 3.29 (s, 3H), 3.19 (dtt, J=4.7, 3.3, 2.0 Hz, 1H), 2.67-2.55 (m, 3H), 2.40-2.32 (m, 2H), 1.93 (dd, J=12.3, 3.9 Hz, 1H), 1.71 (s, 3H).

¹³C NMR (100 MHz, C₆D₆): δ 196.1, 171.8, 143.8, 141.3, 137.8, 132.8, 131.8, 129.4, 128.0, 125.1, 124.1, 123.4, 121.6, 121.2, 114.3, 113.6, 108.7, 58.2, 56.8, 53.0, 47.9, 37.6, 23.0, 21.1.

IR (Neat Film, NaCl): 3060, 2934, 2841, 1681, 1622, 1504, 1347, 1287, 1226, 1177, 1106, 1016, 967, 898, 856, 813, 732, 662, 623 cm⁻¹.

HRMS (ESI+): m/z calc'd for C₂₆H₂₃N₂O₄S₂ [M+H]⁺: 491.1094, found 491.1098.

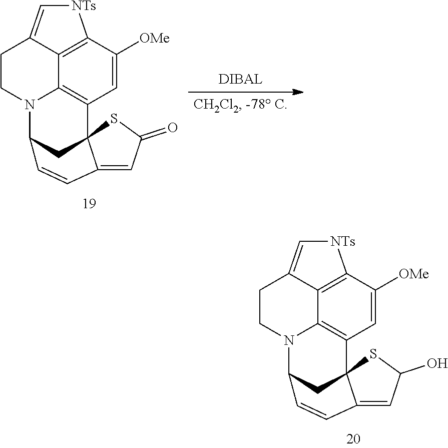

### Thiolactol 20

To a 20 mL glass vial containing diene 19 (20 mg, 0.0408 mmol, 1.0 equiv) under nitrogen was added CH₂Cl₂ (1.9 mL). The solution was cooled to −78° C. in a dry ice/acetone bath. To the resulting mixture was added DIBAL (70 μL, 0.408 mmol, 10 equiv) as a freshly prepared solution in CH₂Cl₂ (1 mL). The reaction was quenched at −78° C. by the addition of 2 mL saturated aqueous Rochelle's salt solution immediately following the addition of DIBAL. The resulting mixture was allowed to warm up to 23° C. and was stirred vigorously for 45 min. The layers were then separated, and the aqueous phase was extracted with CH₂Cl₂ (3×2 mL). The combined organic phases were dried over Na₂SO₄ and concentrated in vacuo to afford crude thiolactol 20 as a yellow oil, which was used immediately in next step without further purification.

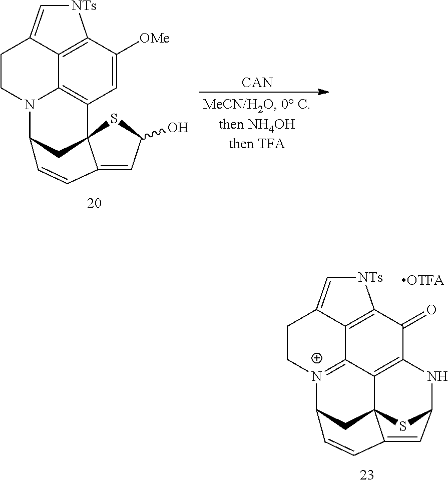

N-tosyl desbromoaleutianamine (23)

To a 20 mL vial containing thiolactol 20 (20 mg, 0.0408 mmol, 1.0 equiv) under nitrogen was added MeCN (2.7 mL). The vial was cooled to 0° C. in an ice bath and CAN (44.8 mg, 0.0816 mmol, 2 equiv) was added dropwise as a freshly prepared solution in 1.3 mL water. The resulting mixture was stirred at 0° C. for 5 min, after which aqueous ammonia (27% w/w, 34 μL, 0.408 mmol, 10 equiv) was added. The reaction vessel was flushed with 02, and the resulting mixture was stirred for 7 min under an 02 atmosphere at 0° C. The reaction mixture was warmed to 23° C. and was diluted with EtOAc (5 mL) and water (3 mL). The layers were separated, and the aqueous phase was extracted with EtOAc (4×4 mL). The combined organic phases were dried over Na₂SO₄ and concentrated in vacuo. The residue was taken up in TFA (4 mL), and the mixture was allowed to stand at 23° C. for 10 min before being concentrated by rotary evaporation. The crude N-tosyl desbromoaleutianamine (23) was purified by preparative reverse phase HPLC (Eclipse XDB-C₁₈, m, 9.4×250 mm, MeCN—H₂O (0.1% TFA)=32.5:67.5 to 100:0, linear gradient for 6 min, flow rate=5 mL/min) to afford N-tosyl desbromoaleutianamine (23) as the TFA salt (12 mg, 0.0204 mmol, 50% yield).

¹H NMR (400 MHz, CD₃OD): δ 8.09-8.01 (m, 2H), 7.78 (d, J=1.4 Hz, 1H), 7.42 (d, J=8.0 Hz, 2H), 6.72 (d, J=9.4 Hz, 1H), 6.16 (dd, J=9.5, 5.9 Hz, 1H), 5.94 (d, J=3.3 Hz, 1H), 5.31 (d, J=3.3 Hz, 1H), 4.95 (dt, J=6.0, 2.9 Hz, 1H), 4.26-4.16 (m, 1H), 4.15-3.97 (m, 1H), 3.22 (td, J=15.4, 7.1 Hz, 1H), 3.16-3.04 (m, 1H), 2.51-2.31 (m, 5H).

¹³C NMR (100 MHz, MeOD): δ 167.4, 148.7, 147.4, 141.9, 140.7, 134.6, 131.1, 130.1, 129.3, 127.8, 124.4, 123.7, 118.8, 112.3, 102.5, 64.8, 57.6, 51.9, 50.9, 30.9, 21.7, 20.3. (Note: two ¹³C signals overlap.) IR (Neat Film, NaCl): 3136, 2932, 1682, 1622, 1524, 1442, 1420, 1378, 1346, 1268, 1194, 1180, cm¹.

HRMS (ESI+): m/z calc'd for C₂₅H₂₀N₃O₃S₂[M+H]⁺: 474.0941, found 474.0959.

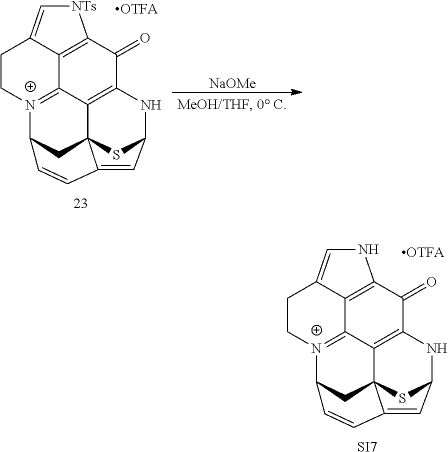

### Desbromoaleutianamine (SI7)

To a 4 mL vial containing N-tosyl desbromoaleutianamine (23, 12 mg, 0.0204 mmol, 1.0 equiv) under nitrogen was added THF (1.2 mL). The resulting mixture was cooled to 0° C. with an ice water bath, and 0.2 μM NaOMe in MeOH (0.72 mL, 0.14 mmol, 7.0 equiv) was added. The reaction mixture was stirred at 0° C. for 10 min, after which 0.2 μM TFA in MeOH (0.72 mL, 0.144 mol, 7.0 equiv) was added. The reaction mixture was allowed to warm to 23° C. The mixture was then concentrated in vacuo and the residue was purified by preparative reverse-phase HPLC (Eclipse XDB-C18, 5 μm, 9.4×250 mm, MeCN—H₂O (0.1% TFA)=0:100 to 100:0, linear gradient for 6 min, flow rate=5 mL/min) to afford desbromoaleutianamine (SI7) as the TFA salt (7 mg, 0.0162 mmol, 79% yield).

¹H NMR (600 MHz, CD₃OD): δ 7.06 (d, J=1.0 Hz, 1H), 6.74 (d, J=9.4 Hz, 1H), 6.23 (dd, J=9.5, 5.9 Hz, 1H), 5.95 (d, J=3.4 Hz, 1H), 5.35 (d, J=3.4 Hz, 1H), 4.92 (dt, J=6.0, 2.9 Hz, 1H), 4.18 (ddd, J=14.2, 7.3, 2.0 Hz, 1H), 4.04 (td, J=14.1, 6.4 Hz, 1H), 3.22 (dddd, J=15.4, 14.0, 7.2, 1.2 Hz, 1H), 3.06 (ddd, J=16.4, 6.4, 2.0 Hz, 1H), 2.52 (ddd, J=12.7, 2.8, 1.5 Hz, 1H), 2.43 (dd, J=12.7, 3.0 Hz, 1H).

¹³C NMR (100 MHz, CD₃OD): δ 169.0, 149.2, 142.0, 141.5, 127.4, 126.3, 125.7, 124.3, 122.6, 119.0, 112.3, 102.0, 64.8, 56.8, 52.4, 51.1, 31.4, 20.9.

IR (Neat Film, NaCl): 3106, 2930, 1672, 1620, 1580, 1556, 1520, 1486, 1436, 1412, 1376, 1342, 1270, 1202, 1132, 1034, 1000, 964, 824, 798, 720, 676 cm¹.

HRMS (ESI+): m/z calc'd for C₁₈H₁₄N₃OS [M+H]⁺: 320.0852, found 320.0857.

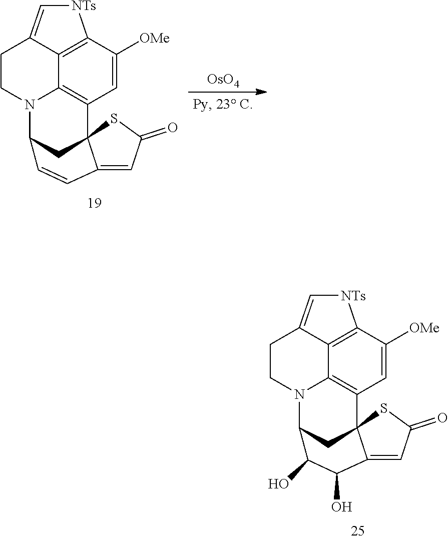

### Diol 25

To a 20 mL glass vial equipped with a septum cap was added diene 19 (20 mg, 0.041 mmol, 1.0 equiv), and the vial was evacuated and backfilled with nitrogen 3 times. Freshly distilled pyridine (1 mL) was added, followed by OsO₄ (11.4 mg, 0.0449 mmol, 1.1 equiv) as a solution in pyridine (0.3 mL). The reaction mixture was stirred at 23° C. for 30 min, after which LC-MS analysis indicated complete consumption of the starting material. The reaction was quenched by addition of saturated aqueous Na₂SO₃ (0.5 mL) and water (5 mL). The mixture was extracted with EtOAc (4×5 mL), and the combined organic layers were dried over Na₂SO₄, filtered, and concentrated in vacuo. The crude osmate ester was dissolved in THF (1.5 mL), then saturated aqueous Na₂SO₃ (0.5 mL) and water (1 mL) were sequentially added. The resulting mixture was vigorously stirred for 15 h before water (5 mL) was added. The mixture was extracted with EtOAc (3×7 mL), dried with Na₂SO₄, filtered, and concentrated in vacuo. The crude product was purified by silica gel flash chromatography (40→80% EtOAc/hexanes) to afford diol 25 as a light-yellow foam (18.9 mg, 0.0361 mmol, 88% yield).

¹H NMR (400 MHz, CD₂Cl₂): δ 7.75 (d, J=8.4 Hz, 2H), 7.31 (s, 1H), 7.27 (d, J=7.7 Hz, 2H), 6.77 (s, 1H), 5.98 (d, J=2.0 Hz, 1H), 4.37 (t, J=3.8 Hz, 1H), 4.26 (dd, J=4.3, 1.9 Hz, 1H), 3.72 (q, J=3.2 Hz, 1H), 3.62 (s, 3H), 3.42 (t, J=5.8 Hz, 2H), 3.07-2.93 (m, 2H), 2.83 (br, 1H), 2.58 (dd, J=12.7, 3.0 Hz, 1H), 2.45 (ddd, J=12.7, 3.0, 1.0 Hz, 1H), 2.37 (s, 3H), 1.64 (br, 1H).

¹³C NMR (100 MHz, CD₂Cl₂): δ 197.1, 178.6, 145.1, 139.7, 137.0, 131.6, 129.8, 127.9, 123.7, 123.1, 122.1, 121.1, 114.7, 112.4, 108.4, 73.7, 69.2, 60.3, 58.1, 57.2, 48.4, 34.6, 23.3, 21.7.

IR (Neat Film, NaCl): 3442, 2924, 1682, 1504, 1450, 1348, 1286, 1230, 1168, 1094, 912, 794, 732, 666 cm¹.

HRMS (ESI+): m/z calc'd for C₂₆H₂₅N₂O₆S₂[M+H]⁺: 525.1154, found 525.1144.

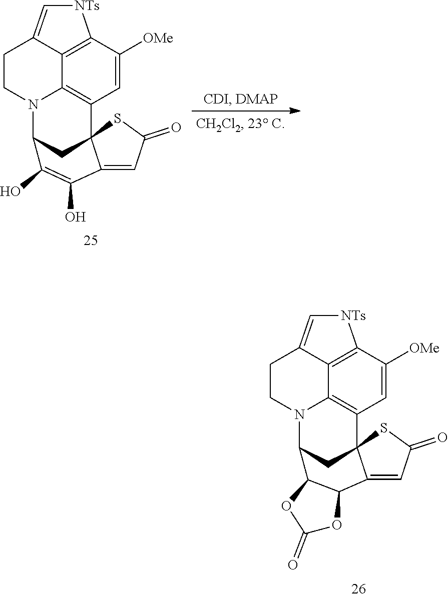

### Carbonate 26

To a 20 mL glass vial equipped with a septum cap was added diol 25 (20.8 mg, 0.0397 mmol, 1.0 equiv) and DMAP (0.1 mg, 0.8 mol, 0.02 equiv). The vial was evacuated and backfilled with nitrogen 3 times. CDI (18.6 mg, 0.119 mmol, 3.0 equiv) was added as a solution in CH₂Cl₂ (4 mL). The reaction mixture was stirred at 23° C. for 30 min, after which TLC analysis indicated full consumption of starting material. The reaction mixture was washed with 5% aqueous citric acid (3×5 mL), dried with Na₂SO₄, and concentrated in vacuo. The crude product was purified by silica gel flash chromatography (0->35% EtOAc/hexanes) to afford cyclic carbonate 26 as a yellow foam (20 mg, 0.0363 mmol, 92%).

¹H NMR (400 MHz, C₆D₆): δ 7.88-7.80 (m, 2H), 7.46 (d, J=1.2 Hz, 1H), 6.64-6.62 (m, 2H), 6.61 (s, 1H), 5.97 (d, J=2.0 Hz, 1H), 3.73 (dt, J=6.7, 1.8 Hz, 1H), 3.59 (ddd, J=6.6, 2.1, 0.7 Hz, 1H), 3.28 (s, 3H), 2.92 (q, J=2.8 Hz, 1H), 2.49-2.27 (m, 4H), 1.96 (ddd, J=13.4, 2.7, 1.4 Hz, 1H), 1.74 (dd, J=13.4, 3.5 Hz, 1H), 1.70 (s, 3H).

¹³C NMR (100 MHz, C₆D₆): δ 193.9, 170.4, 152.1, 144.3, 141.2, 137.5, 130.7, 129.5, 124.8, 124.3, 122.5, 121.5, 113.6, 109.5, 108.3, 75.9, 73.3, 57.8, 56.7, 53.0, 47.4, 34.1, 22.7, 21.1. (Note: an additional ¹³C resonance associated with the tosyl group is likely obscured by the solvent signal.)

IR (Neat Film, NaCl): 2920, 2843, 1832, 1814, 1690, 1504, 1440, 1404, 1354, 1288, 1230, 1168, 1138, 1106, 1056, 838, 814, 666 cm¹.

HRMS (ESI+): m/z calc'd for C₂₇H₂₃N₂O₇S₂ [M+H]⁺: 551.0941, found 551.0937.

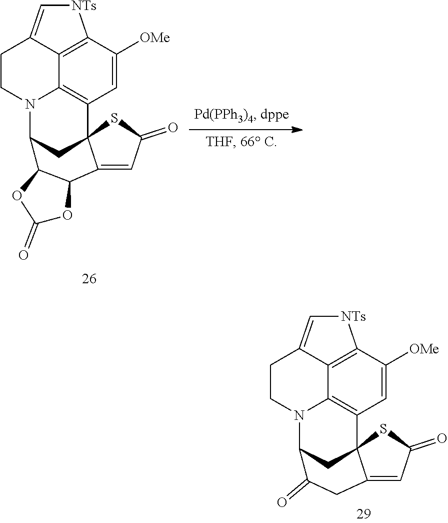

### Ketone 29

Procedure adopted from known literature methods. A 20 mL glass vial containing cyclic carbonate 26 (20.6 mg, 0.0375 mmol, 1.0 equiv) was transferred to a nitrogen-filled glovebox. In a separate vial, a stock solution of Pd(PPh₃)₄ (7.7 mg) and dppe (2.7 mg) in THF (2.6 mL) was prepared and stirred at 28° C. for 10 min. Then, 1.5 mL of the catalyst solution was transferred to the vial containing cyclic carbonate 26 (for 4.4 mg of Pd(PPh₃)₄[0.00375 mmol, 10 mol %] and 1.5 mg dppe [0.00375 mmol, 10 mol %]). The vial was sealed with a PTFE/silicone septum cap and electrical tape, removed from the glovebox, and stirred at 66° C. for 5 h. The reaction mixture was concentrated in vacuo and purified by silica gel flash chromatography (0->40% EtOAc/hexanes) to afford ketone 29 as a yellow oil (18 mg, 0.036, 94% yield).

¹H NMR (400 MHz, C₆D₆): δ 7.88-7.79 (m, 2H), 7.42 (s, 1H), 6.80 (s, 1H), 6.62 (d, J=8.1 Hz, 2H), 5.20 (d, J=2.2 Hz, 1H), 3.29 (s, 3H), 3.17-3.10 (m, 1H), 2.86 (dd, J=17.0, 1.6 Hz, 1H), 2.73-2.64 (m, 1H), 2.60-2.43 (m, 2H), 2.38-2.25 (m, 2H), 2.11 (dd, J=17.0, 2.4 Hz, 1H), 1.72 (s, 3H), 1.51 (dd, J=13.3, 3.2 Hz, 1H).

¹³C NMR (100 MHz, C₆D₆): δ 198.5, 195.5, 172.9, 144.1, 141.3, 137.7, 130.3, 129.4, 124.4, 122.9, 122.6, 121.7, 113.8, 112.0, 108.0, 62.9, 58.7, 56.7, 48.1, 43.9, 35.2, 22.6, 21.1. (Note: an additional ¹³C resonance associated with the tosyl group is likely obscured by the solvent signal.)

IR (Neat Film, NaCl): 2922, 1722, 1688, 1634, 1596, 1502, 1434, 1350, 1286, 1228, 1172, 1106, 1010, 968, 894, 850, 830, 814, 718, 680, 664, 650, 612 cm⁻¹.

HRMS (ESI+): m/z calc'd for C₂₆H₂₃N₂O₅S₂ [M+H]⁺: 507.1043, found 507.1051.

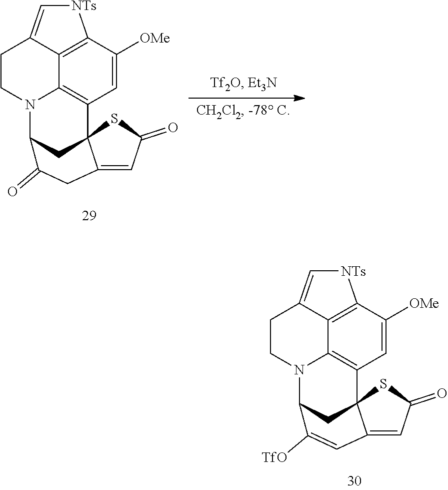

### Enol Triflate 30

To a 20 mL glass vial containing ketone 29 (12 mg, 0.024 mmol, 1.0 equiv) under nitrogen was added CH₂Cl₂ (2.4 mL). The mixture was cooled to −78° C. in a dry ice/acetone bath, then triethyl amine (34 μL, 0.24 mmol, 10 equiv) and triflic anhydride (20 μL, 0.12 mmol, 5.0 equiv) were sequentially added. The reaction mixture was stirred at −78° C. and the reaction progress was monitored by LC-MS. The reaction was quenched by addition of water (2 mL) at −78° C. upon completion (ca. 5-10 min). The resulting mixture was allowed to warm up to 23° C., then the layers were separated. The organic phase was washed with water (2×2 mL), dried with Na₂SO₄, and concentrated in vacuo. The crude product was purified by silica gel flash chromatography (0->10% EtOAc/CH₂Cl₂) to afford enol triflate 30 as a red film (14.2 mg, 0.0222 mmol, 94% yield).

¹H NMR (400 MHz, C₆D₆): δ 7.83-7.75 (m, 2H), 7.41 (d, J=1.7 Hz, 1H), 6.75 (s, 1H), 6.61-6.53 (m, 2H), 5.71 (s, 1H), 5.28 (s, 1H), 3.49 (ddd, J=3.8, 2.5, 1.1 Hz, 1H), 3.29 (s, 3H), 2.82-2.72 (m, 2H), 2.52 (dddd, J=15.7, 10.8, 6.7, 2.0 Hz, 1H), 2.31 (dt, J=15.7, 2.9 Hz, 1H), 2.15 (dd, J=12.5, 2.5 Hz, 1H), 1.79 (dd, J=12.5, 3.9 Hz, 1H), 1.67 (s, 3H).

¹³C NMR (100 MHz, C₆D₆): δ 195.1, 167.5, 150.2, 144.1, 141.9, 137.5, 130.4, 129.4, 128.0, 124.8, 124.3, 123.5, 121.8, 118.9 (q, J=322.2 Hz), 116.3, 114.1, 112.1, 108.1, 56.8, 56.6, 56.5, 49.2, 36.4, 22.8, 21.0.

IR (Neat Film, NaCl): 3310, 3064, 2918, 2852, 1694, 1682, 1634, 1598, 1538, 1504, 1424, 1366, 1350, 1326, 1284, 1216, 1188, 1176, 1136, 1110, 1062, 968, 940, 900, 822, 760, 672, 664, 630, 610 cm⁻¹.

HRMS (ESI+): m/z calc'd for C₂₇H₂₁F3N₂O₇S₃ [M]⁺: 638.0463, found 638.0484.

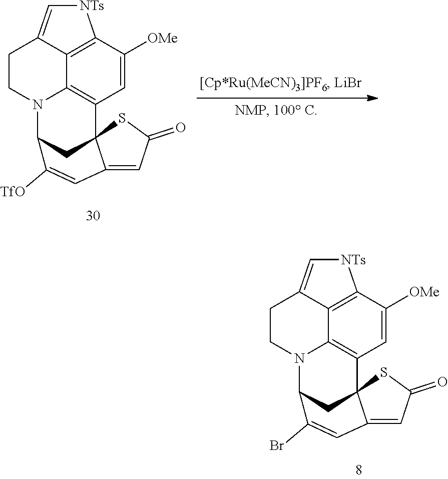 [SMILES: COc1cc2c3c4c(cn(S(=O)(=O)c5ccc(C)cc5)c14)CCN3[C@@H]1C[C@]23SC(=O)C=C3C=C1OS(=O)(=O)C(F)(F)F ; COc1cc2c3c4c(cn(S(=O)(=O)c5ccc(C)cc5)c14)CCN3[C@@H]1C[C@]23SC(=O)C=C3C=C1Br]

### Alkenyl Bromide 8

A 4 mL vial containing enol triflate 30 (18 mg, 0.028 mmol, 1.0 equiv) was brought into a nitrogen-filled glovebox. In a separate vial, a stock solution of [Cp*Ru(MeCN)₃]PF₆ (10.2 mg) and LiBr (17.7 mg) in NMP (2.3 mL) was prepared and stirred at 28° C. for 10 mins. Then, 0.94 mL of the catalyst solution (for 4.3 mg of [Cp*Ru(MeCN)₃]PF_{6 [0.00852} mmol, 30 mol %] and 7.3 mg LiBr [0.0852 mmol, 3 equiv]) was transferred to the vial containing enol triflate 30. The vial was sealed with a PTFE/silicone septum cap and electrical tape, removed from glovebox, and stirred at 100° C. for 5 h. Then the reaction mixture was diluted with Et₂O (2 mL) and water (2 mL) and filtered through a PTFE syringe filter. The layers were separated, and the aqueous layer was extracted with Et₂O (4×2 mL). The combined organic phased were dried over Na₂SO₄ and concentrated in vacuo. The crude product was purified by silica gel flash chromatography (100% hexane→100% CH₂Cl₂) to afford alkenyl bromide 8 (6.8 mg, 0.012 mmol, 38% yield) as an orange film.

¹H NMR (400 MHz, C₆D₆): δ 7.84-7.76 (m, 2H), 7.41 (d, J=1.7 Hz, 1H), 6.83 (s, 1H), 6.61-6.53 (m, 2H), 6.15 (s, 1H), 5.34 (s, 1H), 3.48-3.42 (m, 1H), 3.29 (s, 3H), 3.17 (ddd, J=12.2, 11.1, 3.7 Hz, 1H), 2.90 (ddd, J=11.1, 4.9, 2.5 Hz, 1H), 2.55 (dddd, J=15.6, 12.1, 4.9, 1.9 Hz, 1H), 2.40 (dt, J=15.6, 3.2 Hz, 1H), 2.14 (dd, J=12.3, 2.5 Hz, 1H), 1.83-1.73 (m, 1H), 1.70 (s, 3H).

¹³C NMR (100 MHz, C₆D₆): δ 195.5, 170.3, 143.9, 141.6, 137.7, 130.7, 129.4, 127.6, 124.3, 123.3, 121.4, 121.1, 114.2, 112.1, 108.4, 61.4, 56.8, 56.5, 49.7, 38.3, 23.2, 21.1. (Note: additional ¹³C resonances associated with the tosyl group and the alkenyl bromide carbon are likely obscured by the solvent signal.)

IR (Neat Film, NaCl): 3312, 2920, 2850, 2354, 1682, 1632, 1502, 1348, 1170, 1106, 894, 804, 662 cm⁻¹.

HRMS (ESI+): m/z calc'd for C₂₆H₂₂BrN₂O₄S₂ [M+H]⁺: 571.0184, found 571.0154.

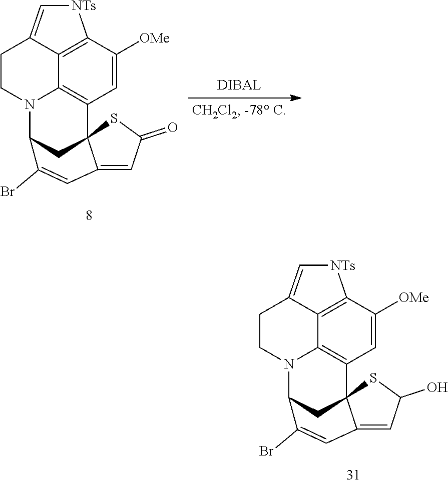

### Thiolactol 31

To an 8 mL vial containing alkenyl bromide 8 (6.8 mg, 0.012 mmol, 1.0 equiv) under nitrogen was added CH₂Cl₂ (0.9 mL). The resulting mixture was cooled to −78° C. in a dry ice/acetone bath, then DIBAL (21 μL, 0.12 mmol, 10 equiv) was added as a freshly prepared solution in CH₂Cl₂ (0.3 mL, 0.4 M). The reaction was quenched at −78° C. by addition of 1 mL saturated aqueous Rochelle's salt solution immediately after the addition of DIBAL. The resulting mixture was allowed to warm up to 23° C. and was stirred vigorously for 45 min. The layers were separated and the aqueous phase was extracted with CH₂Cl₂ (3×1 mL). The combined organic phases were dried over Na₂SO₄ and concentrated in vacuo to afford crude thiolactol 31 as a yellow oil, which was used directly in the next step without further purification.

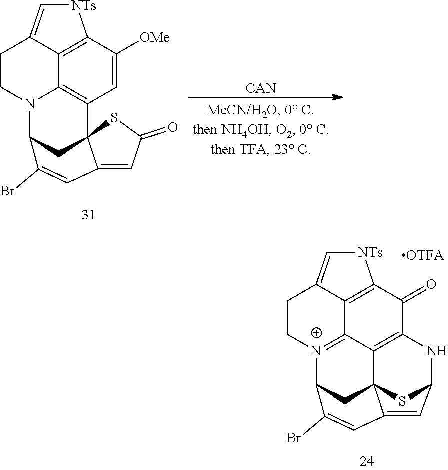 [SMILES: COc1cc2c3c4c(cn(S(=O)(=O)c5ccc(C)cc5)c14)CCN3[C@@H]1C[C@]23SC(=O)C=C3C=C1Br ; "CAN" ; Cc1ccc(S(=O)(=O)n2cc3c4c2C(=O)C2=C5C4=[N+](CC3)[C@@H]3C[C@]54S[C@@H](C=C4C=C3Br)N2)cc1]

### N-Tosyl Aleutianamine (24)

To an 8 mL vial containing crude thiolactol 31 (6.8 mg, 0.012 mmol, 1.0 equiv) under nitrogen was added MeCN (0.8 mL). The resulting solution was cooled to 0° C. in an ice water bath, and CAN (13.2 mg, 0.024 mmol, 2.0 equiv) was added as a freshly prepared solution in 0.3 mL of water. The resulting mixture was stirred at 0° C. for 5 min, whereafter concentrated aqueous ammonia (27% w/w, 35 μL, 0.42 mmol, 35 equiv) was added. The reaction vessel was flushed with 02, and the resulting mixture was stirred for 7 min under an 02 atmosphere at 0° C. The reaction mixture was then warmed to 23° C. and was diluted with EtOAc (2 mL) and water (1.5 mL). The layers were separated, and the aqueous phase was extracted with EtOAc (4×2 mL). The combined organic phases were dried over Na₂SO₄ and concentrated in vacuo. The residue was taken up in trifluoroacetic acid (1 mL), and the mixture was allowed to stand at 23° C. for 10 min before being concentrated in vacuo. The crude N-tosyl aleutianamine (24) was used directly in the next step without further purification.

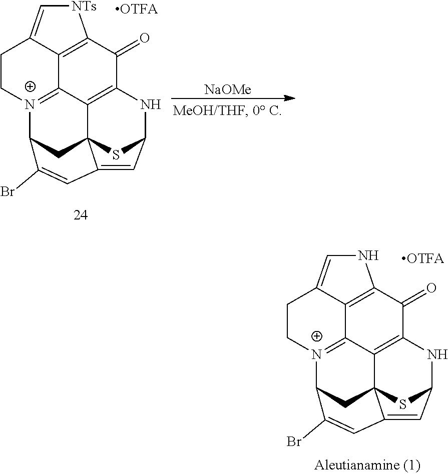 [SMILES: Cc1ccc(S(=O)(=O)n2cc3c4c2C(=O)C2=C5C4=[N+](CC3)[C@@H]3C[C@]54S[C@@H](C=C4C=C3Br)N2)cc1 ; O=C1C2=C3C4=[N+](CCc5c[nH]c1c54)[C@@H]1C[C@]34S[C@@H](C=C4C=C1Br)N2 ; "NaOMe"]

### Aleutianamine (1)

Procedure adapted from known literature methods. To a 4 mL vial containing crude N-tosyl aleutianamine (2, 0.012 mmol, 1.0 equiv) under nitrogen was added THF (0.78 mL). The resulting mixture was cooled to 0° C. in an ice bath and NaOMe in MeOH (0.2 M, 0.42 mL, 0.084 mmol, 7 equiv) was added. The reaction mixture was stirred at 0° C. for 10 min, after which TFA in MeOH (0.2 M, 0.42 mL) was added and the reaction mixture was allowed to warm to 23° C. The mixture was concentrated in vacuo and the residue was purified by preparative reverse-phase HPLC (Eclipse XDB-C18, 5 μm, 9.4×250 mm, MeCN—H₂O (0.1% TFA)=0:100 to 100:0, linear gradient for 6 min, flow rate=5 mL/min) to afford aleutianamine (1) as TFA salt (2 mg, 0.00391 mmol, 33% over three steps).

¹H NMR (400 MHz, CD₃OD): δ 7.14 (s, 1H), 7.08 (d, J=1.1 Hz, 1H), 5.97 (d, J=3.4 Hz, 1H), 5.43-5.38 (m, 1H), 5.13 (t, J=3.0 Hz, 1H), 4.29-4.22 (m, 2H), 3.29-3.18 (m, 1H), 3.14-3.05 (m, 1H), 2.67 (dd, J=12.8, 3.1 Hz, 1H), 2.57 (ddd, J=12.8, 3.0, 1.2 Hz, 1H).

¹³C NMR (100 MHz, CD₃OD): δ 168.6, 149.8, 142.9, 142.2, 129.2, 126.5, 125.8, 122.7, 119.3, 118.1, 112.5, 101.8, 65.4, 64.9, 54.1, 49.8, 32.5, 21.2.

IR (Neat film, NaCl): 3106, 2928, 1674, 1616, 1520, 1410, 1342, 1170, 996, 800,720 cm¹.

HRMS (ESI+): m/z calc'd for C₁₈H₁₃BrN₃OS [M+H]⁺: 399.9937, found 399.9945.

### Example 2: Characterization of Synthetic Aleutianamine (1)

TABLE S1
¹H NMR comparison table for (−)-aleutianamine (1)

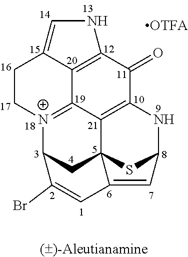 [SMILES: O=C1C2=C3C4=[N+](CCc5c[nH]c1c54)[C@@H]1C[C@]34S[C@@H](C=C4C=C1Br)N2]

| | Natural aleutianamine | Reported Synthetic | Synthetic aleutianamine |
|---|---|---|---|
| | TFA salt (600 MHz, | aleutianamine TFA | TFA salt, this work, (500 |
| | CD₃OD) | salt,⁶ (600 MHz, | MHz, CD₃OD) |
| H-1 | 7.12 (s, 1H) | 7.14 (s, 1H) | 7.14 (s, 1H) |
| H-14 | 7.07 (s, 1H) | 7.09 (s, 1H) | 7.08 (d, J = 1.1 Hz, 1H) |
| H-8 | 5.96 (d, J = 3.3 Hz, 1H) | 5.97 (d, J = 3.0 Hz, 1H) | 5.97 (d, J = 3.4 Hz, 1H) |
| H-7 | 5.40 (d, J = 3.2 Hz, 1H) | 5.41 (d, J = 3.0 Hz, 1H) | 5.43-5.38 (m, 1H) |
| H-3 | 5.12 (t, J = 2.9 Hz, 1H) | 5.13 (t, J = 3.0 Hz, 1H) | 5.13 (t, J = 3.0 Hz, 1H) |
| H-17A | 4.26 (dd, J = 10.5, 4.3 Hz, 1H) | 4.28-4.23 (m, 2H) | 4.29-4.22 (m, 2H) |
| H-17B | 4.24 (dd, J = 10.5, 4.3 Hz, 1H) | | |
| H-16A | 3.22 (m, 1H) | 3.25-3.18 (m, 1H) | 3.29-3.18 (m, 1H) |
| H-16B | 3.10 (dt, J = 16.2, 4.2 Hz, 1H) | 3.12-3.06 (m, 1H) | 3.14-3.05 (m, 1H) |
| H-4A | 2.65 (dd, J = 12.7, 2.2 Hz, | 2.66 (dd, J = 12.6, 3.0 | 2.67 (dd, J = 12.8, |
| | 1H) | Hz, 1H) | 3.1 Hz, 1H) |
| H4B | 2.54 (dd, J = | 2.56 (dd, J = | 2.57 (ddd, J = 12.8, |
| | 12.7, 3.0 Hz, 1H) | 12.6, 3.0 Hz, 1H) | 3.0, 1.2 Hz, 1H) |

TABLE S2
¹³C NMR comparison table (−)-aleutianamine (1)

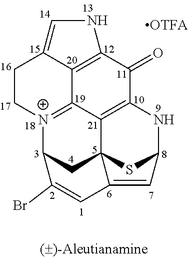 [SMILES: O=C1C2=C3C4=[N+](CCc5c[nH]c1c54)[C@@H]1C[C@]34S[C@@H](C=C4C=C1Br)N2]

| | Natural | Reported | Synthetic | Deviation |
|---|---|---|---|---|
| | aleutianamine | Synthetic | aleutianamine | (synthetic)- |
| | TFA salt (150 | aleutianamine | TFA salt, this | (natural) Δδ |
| C-11 | 167.2 | 168.6 | 168.6 | 1.4 |
| C-19 | 148.3 | 149.8 | 149.8 | 1.5 |
| C-6 | 141.5 | 142.9 | 142.9 | 1.4 |
| C-10 | 140.8 | 142.2 | 142.2 | 1.4 |
| C-1 | 127.8 | 129.2 | 129.2 | 1.4 |
| C-14 | 125.2 | 126.5 | 126.5 | 1.3 |
| C-12 | 124.3 | 125.8 | 125.8 | 1.5 |
| C-15 | 121.2 | 122.7 | 122.7 | 1.5 |
| C-20 | 118.0 | 119.3 | 119.3 | 1.3 |
| C-2 | 116.7 | 118.1 | 118.1 | 1.4 |
| C-7 | 111.1 | 112.5 | 112.5 | 1.4 |
| C-21 | 100.3 | 101.8 | 101.8 | 1.5 |
| C-8 | 64.0 | 65.4 | 65.4 | 1.4 |
| C-3 | 63.4 | 64.9 | 64.9 | 1.5 |
| C-17 | 52.6 | 54.1 | 54.1 | 1.5 |
| C-5 | 48.5 | 49.8 | 49.8 | 1.3 |
| C-4 | 31.1 | 32.5 | 32.5 | 1.4 |
| C-16 | 19.8 | 21.2 | 21.2 | 1.4 |

Note: The small difference in ¹H and ³C NMR shift between synthetic and natural sample is likely resulted from incorrect referencing of the solvent signal.

Reaction conditions for the attempted allylic oxidation of S18 are depicted below. The attempted conditions are not exhaustive.

TABLE S3
Attempted conditions for allylic oxidation

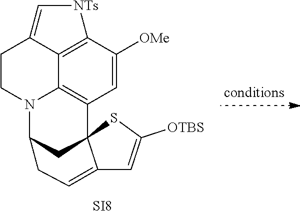

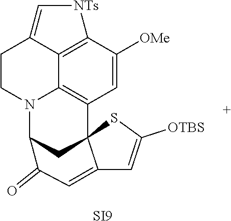

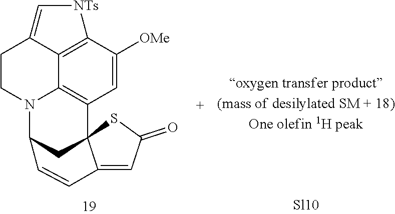

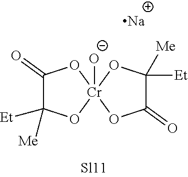

| entry | conditions | result |
|---|---|---|
| 1 | Pd(OH)₂/C, TBHP, Cs₂CO₃, CH₂Cl₂, 0° C. | complex mixture, |
| | | including 19 |
| 2 | PIDA, TBHP, Mg(OAc)₄•4H₂O, n-BuOC(O)n-Pr, 0° C. | complex mixture, |
| | | few olefin peaks |
| | | by NMR |
| 3 | Mn(OAc)₃•2H₂O, TBHP, 4 Á MS, O₂, EtOAc, 23° C. | complex mixture |
| | | by NMR, trace mass |
| | | of Sl9 by LC-MS |
| 4 | Rh₂(esp)₂, TBHP, EtOAc, 23° C. | SM + 2-3 new products, |
| | | Major new product is Sl10 |
| 5 | Rh₂(cap)₄, TBHP, K₂CO₃, CH₂Cl₂, 40° C. | complex mixture; |
| | | tosyl peaks are visible |
| | | but no clear olefinic peaks |
| 6 | Sl10 (10 equiv), MnO₂ (20 equiv), 15-crown-5, DCE, 50° C., 2 d | decomp to baseline |
| 7 | Sl10 (5 equiv), MnO₂ (10 equiv), 15-crown-5, DCE, 80° C., 4.5 h | low yield of 19 |
| 8 | PDC, 4 Á MS, CH₂Cl₂, 60° C. | SM, many oxidized |
| | | products |

Reaction conditions for the attempted bromination of 23 are depicted below. The attempted conditions are not exhaustive.

TABLE S4
Attempted conditions for bromination of S23

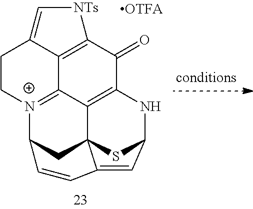 [SMILES: Cc1ccc(S(=O)(=O)n2cc3c4c2C(=O)C2=C5C4=[N+](CC3)[C@H]3C=CC4=C[C@@H](N2)S[C@]45C3)cc1]

| entry | conditions | result |
|---|---|---|
| 1 | NBS, THF, 23° C. | complex mixture |
| 2 | NBS, MeCN, 23° C. | unidentified rearrangement |
| | | product |
| 3 | Br₂, CH₂Cl₂, 23° C. | unidentified rearragement |
| | | product |
| 4 | PyHBr₃, CH₂Cl₂, 23° C. | No reaction |

Various attempts to functionalize the C2 carbon are depicted in Scheme S1 below. The depicted attempts are not exhaustive.

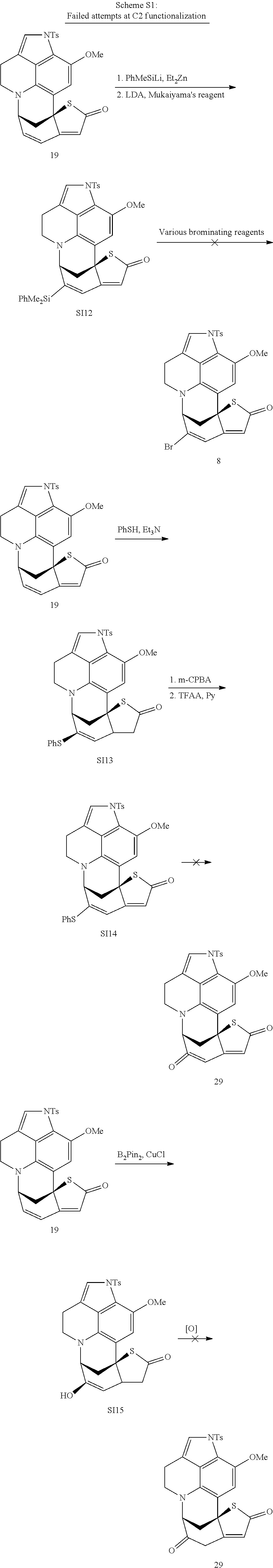

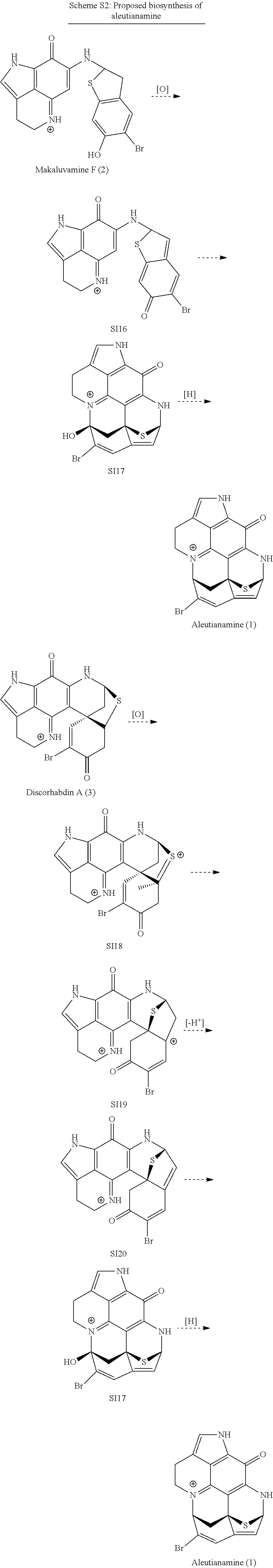

### Example 3: Exemplary Biological Data

Anti-proliferative activity was measured using a CellTiter Glo® Luminescent Cell Viability Assay (Promega G7572) according to the manufacturer's procedure. DMEM containing 5% FBS and 1% Penicillin-Streptomycin was used as cell viability assay medium. Generally, 30 L of 25 cells/L cell suspension was plated in a 384-well plate (Greiner 781080) and compounds was added, final concentration of 55 uM to 0.08 μM by adding 8 μL of media containing 5% DMSO or compound (serial dilutions of 3-fold with a starting concentration at 261 μM, and the plate was incubated for an additional 48 h. Cell viability was measured by adding CellTiter Glo reagent and reading the signal with BioTek Synergy Neo2 plate reader. The IC₅₀ values were calculated from 4 replicates using the percentage of growth of treated cells versus the DMSO control. The results were analyzed using GraphPad Prism 7.0.

TABLE 11
IC₅₀ values of aleutianamine and analogs

| IC50 (uM) | HCT116 | PANC1 | MCF7 | LNCaP | PC3 | DU145 |
|---|---|---|---|---|---|---|
| Compound 1 | 0.07 | 0.09 | 0.001 | 0.0842 | 0.00004 | 0.095 |
| Compound 2 | 0.24 | 0.21 | 0.2 | 0.241 | 0.10 | 0.82 |
| Compound 3 | 29 | >55 | 51.0 | >55 | 34 | 42 |
| Compound 4 | >55 | >55 | >55 | >55 | 59 | >55 |
| Compound 5 | >55 | >55 | >55 | >55 | >55 | >55 |
| Compound 6 | 0.38 | 0.6 | 0.3 | 0.5867 | 0.30 | 1.2 |
| Compound 7 | >55 | >55 | >55 | >55 | >55 | >55 |
| Compound 8 | >55 | >55 | >55 | >55 | >55 | >55 |
| Compound 9 | 25 | 33 | 24 | >55 | 23 | 30 |
| Compound 10 | 7 | 7 | 10 | >55 | 3.5 | 55 |
| Compound 11 | 2 | 3 | 1.7 | 6 | 1.7 | 6 |
| Compound 12 | 12 | 26 | 5.2 | >55 | 4.9 | >55 |
| Compound 13 | 0.3 | 0.74 | 0.20 | 0.84 | 0.55 | 0.008 |
| Compound 14 | 0.1 | 0.21 | 0.09 | 0.31 | 0.24 | 0.009 |
| Compound 15 | >55 | >55 | >55 | 66 | >55 | >55 |
| Compound 16 | >55 | >55 | >55 | >55 | >55 | >55 |

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound having a structure represented by formula I, or a pharmaceutically acceptable salt thereof: 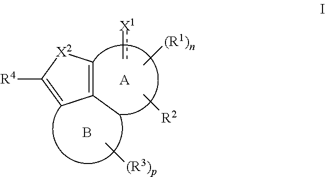
wherein:
each 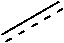
is a single bond or a double bond;
A is aryl, heteroaryl, heterocyclyl, or cycloalkyl;
B is heterocyclyl or cycloalkyl;
R¹, R², R³, and R⁴ are each independently selected from hydrogen, halo, hydroxyl, alkyl, alkenyl, alkynyl, alkoxy, cycloalkyl, cycloalkyl(alkyl), aryl, aralkyl, heterocyclyl, heterocyclylalkyl, heteroaryl, heteroaralkyl, alkylthio, ester, amino, amido, acyl, nitro, cyano, azido, sulfonyl, sulfonamido, and phosphoryl; or
R¹ and R², taken together with the intervening atoms, combine to form an aryl, heteroaryl, heterocyclyl, cycloalkyl, or cycloalkenyl and R² and an instance of R³, taken together with the intervening atoms, combine to form an aryl, heteroaryl, heterocyclyl, cycloalkyl, or cycloalkenyl; or
R² and an instance of R³, taken together with the intervening atoms, combine to form an aryl, heteroaryl, heterocyclyl, cycloalkyl, or cycloalkenyl;
when the bond connected to X¹ is a single bond, X¹ is selected from hydrogen, halo, hydroxyl, alkyl, alkenyl, alkynyl, alkoxy, cycloalkyl, cycloalkyl(alkyl), aryl, aralkyl, heterocyclyl, heterocyclylalkyl, heteroaryl, heteroaralkyl, alkylthio, ester, amino, amido, acyl, nitro, cyano, azido, sulfonyl, sulfonamido, and phosphoryl;
when the bond connected to X¹ is a double bond, X¹ is selected from NR^{j}, O, C(R^{j})₂, and S;
X² is selected from —NR^{j}—, —C(R^{j})₂—, —S(O)—, —S(O)₂—, and —O—;
each R^{j} is independently selected from hydrogen, alkyl, alkenyl, sulfonyl, ester, acyl, aryl, aralkyl, heterocyclyl, heteroaryl, and heteroaralkyl;
n is 1, 2, 3, 4, 5, or 6;
p is 1, 2, 3, 4, 5, 6, 7, 8, or 9; and
wherein the compound is not:

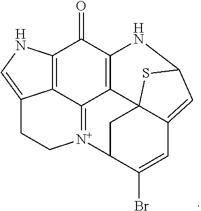 [SMILES: O=C1C2=C3C4=[N+](CCc5c[nH]c1c54)C1CC34SC(C=C4C=C1Br)N2]

2. The compound of claim 1, wherein the compound has a structure represented by formula Ia, or formula Ib or a salt thereof: 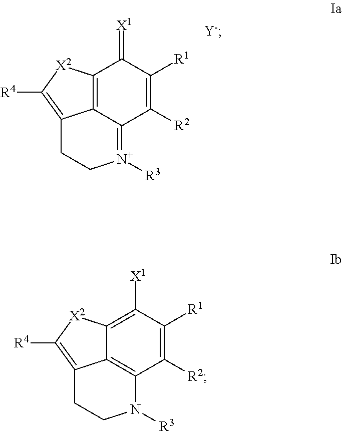
wherein Y⁻ is a counterion.

3. (canceled)

4. The compound of claim 1, wherein X¹ is O.

5. The compound of claim 1, wherein X² is —NR^{j}—.

6. The compound of claim 5, wherein R^{j} is hydrogen, sulfonyl, or alkyl.

7. (canceled)

8. The compound of claim 1, wherein R¹ is amino or azido.

9. (canceled)

10. The compound of claim 1, wherein R⁴ is hydrogen, alkylthio, or alkoxy.

11-12. (canceled)

13. The compound of claim 1, wherein R² is hydrogen or halo.

14. (canceled)

15. The compound of claim 1, wherein R³ is hydrogen, alkyl, or ester.

16-17. (canceled)

18. The compound of claim 1, wherein R² and an instance of R³, taken together with the intervening atoms, combine to form an aryl, heteroaryl, heterocyclyl, cycloalkyl, or cycloalkenyl.

19. The compound of claim 18, wherein the compound has a structure represented by formula Ic, or formula Id or a salt thereof: 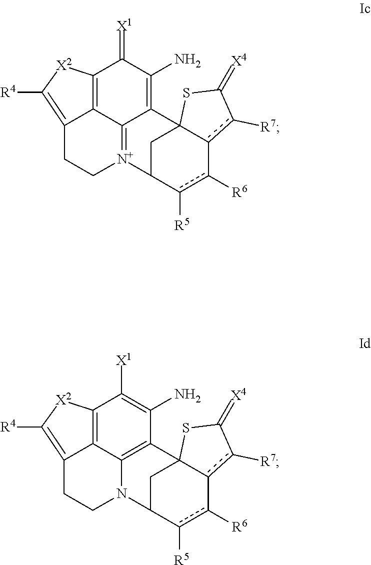
wherein:
X⁴ is NR^{j}, O, or C(R^{j})₂; and
R⁵, R⁶, and R⁷ are each independently selected from hydrogen, halo, hydroxyl, alkyl, alkenyl, alkynyl, alkoxy, cycloalkyl, cycloalkyl(alkyl), aryl, aralkyl, heterocyclyl, heterocyclylalkyl, heteroaryl, heteroaralkyl, alkylthio, ester, amino, amido, acyl, nitro, cyano, azido, sulfonyl, sulfonamido, and phosphoryl.

20-21. (canceled)

22. The compound of claim 1, wherein:
R¹ and R², taken together with the intervening atoms, combine to form an aryl, heteroaryl, heterocyclyl, cycloalkyl, or cycloalkenyl, and R² and an instance of R³, taken together with the intervening atoms, combine to form an aryl, heteroaryl, heterocyclyl, cycloalkyl, or cycloalkenyl.

23. The compound of claim 22, wherein the compound has a structure represented by formula Ie, or formula If or a salt thereof:

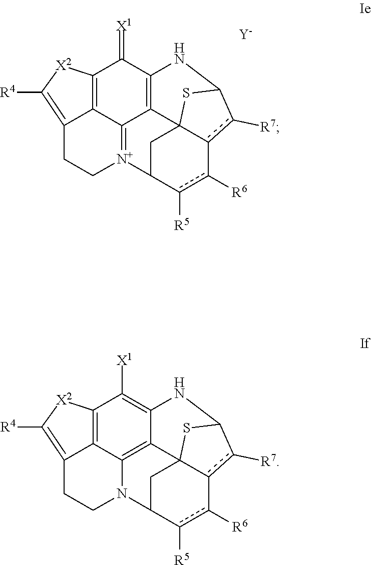

24. (canceled)

25. The compound of claim 22, wherein the compound has a structure represented by formula Ig, or formula Ih or a salt thereof:

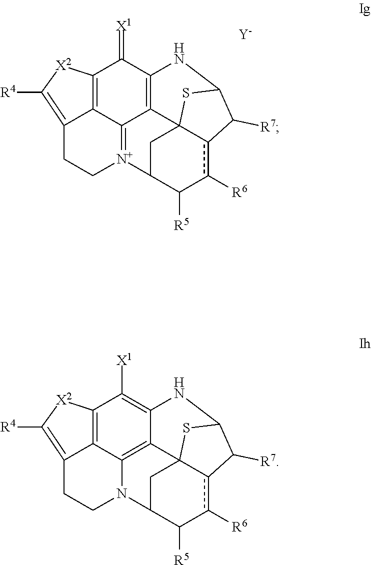

26. (canceled)

27. The compound of claim 1, wherein the compound has a structure represented by formula Ii, or formula Ij or a salt thereof: 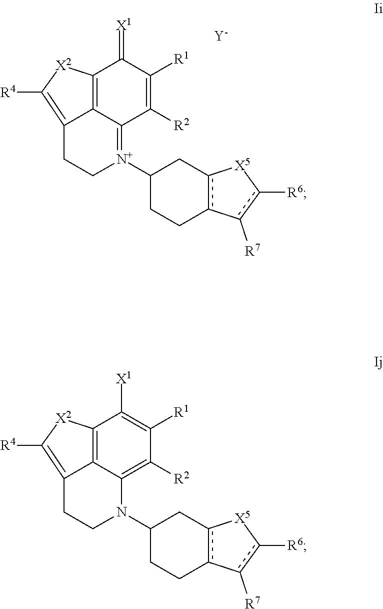
wherein:
X⁵ is —O—, —NH— or —S—; and
R⁶ is selected from hydrogen, halo, alkyl, alkenyl, alkynyl, alkoxy, cycloalkyl, cycloalkyl(alkyl), aryl, aralkyl, heterocyclyl, heterocyclylalkyl, heteroaryl, heteroaralkyl, alkylthio, amino, amido, and nitro.

28-30. (canceled)

31. The compound of claim 1, wherein the compound is selected from: 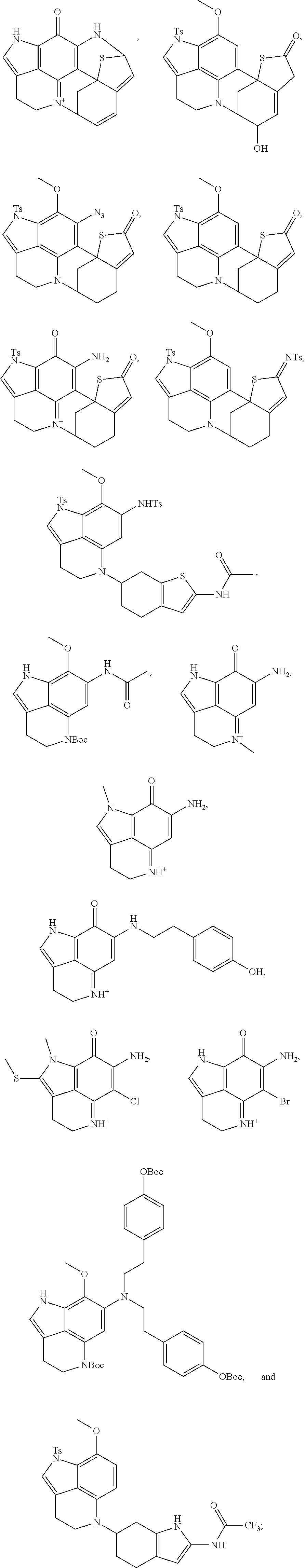
or a pharmaceutically acceptable salt thereof.

32. (canceled)

33. A pharmaceutical composition comprising a compound of claim 1 and a pharmaceutically acceptable excipient.

34. A method of treating a cancer in a subject in need thereof, comprising administering to the subject a compound of claim 1.

35-37. (canceled)

38. A method of making a compound of claim 1, or a salt thereof, wherein the method comprises the step set forth in Scheme I: 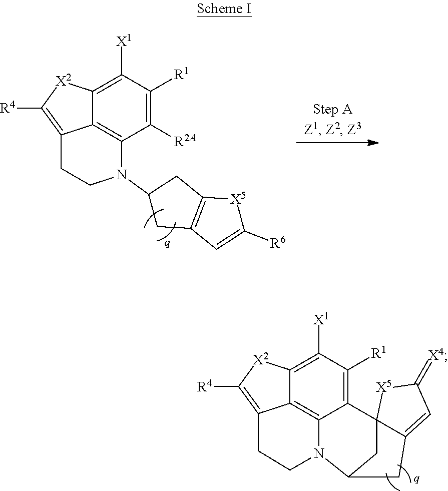
wherein:
R^{2A} is halo;
q is 0, 1, 2, 3, or 4;
Z¹ is a transition metal salt or a transition metal complex;
Z² is a ligand; and
Z³ is a base.

39-55. (canceled)

56. A method of making a compound of claim 1, or a salt thereof, wherein the method comprises the steps set forth in Scheme III: 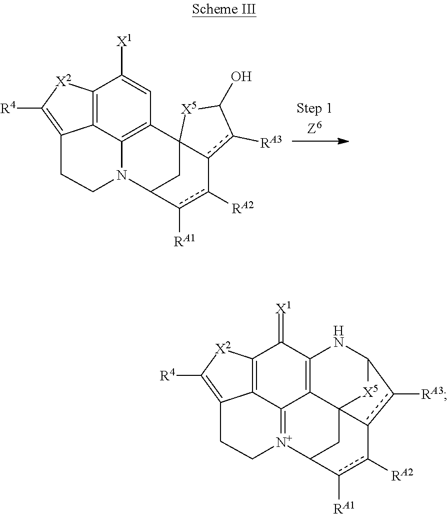
wherein:
each [symbol] is a single bond or a double bond;
R^{A1}, R^{A2}, and R^{A3} are each independently selected from hydrogen, halo, hydroxyl, alkyl, alkenyl, alkynyl, alkoxy, cycloalkyl, cycloalkyl(alkyl), aryl, aralkyl, heterocyclyl, heterocyclylalkyl, heteroaryl, heteroaralkyl, alkylthio, ester, amino, amido, acyl, nitro, cyano, azido, sulfonyl, sulfonamido, and phosphoryl; and
Z⁶ is an oxidizing agent.

57-61. (canceled)
